(19) United States
(12) Patent Application Publication
(10) Pub. No.: US 2006/0159655 A1
(43) Pub. Date: Jul. 20, 2006

(54) Treating immunological disorders using agonists of interleukin-21 / interleukin-21 receptor

(75) Inventors: Mary Collins, Natick, MA (US); Elaine Y. Chin, Concord, MA (US); Mayra Senices, Peabody, MA (US); Deborah A. Young, Melrose, MA (US)

Correspondence Address: FITZPATRICK CELLA (WYETH), 30 ROCKEFELLER PLAZA, NEW YORK, NY, 10112-3800, US

(73) Assignee: Wyeth, Madison, NJ (US)

(21) Appl. No.: 10/806,611

(22) Filed: Mar. 22, 2004

Related U.S. Application Data
(60) Provisional application No. 60/456,920, filed on Mar. 21, 2003.

(51) Int. Cl. A61K 38/21; A61K 39/395; A61K 31/525; A61K 31/4745; A61K 38/20

(52) U.S. Cl. 424/85.2; 424/143.1; 424/85.5; 424/85.6; 424/85.7; 514/291; 514/251

(57) ABSTRACT

Methods and compositions for modulating interleukin-21 (IL-21)/IL-21 receptor (MU-1) activity using agonists of IL-21 or IL-21 receptor (“IL-21R” or “MU-1”), are disclosed. IL-21/IL-21R agonists can be used by themselves or in combination with anti-inflammatory agents to treat, e.g., ameliorate, symptoms associated with immunological disorders of the nervous system, e.g., multiple sclerosis.

### CROSS-REFERENCE TO RELATED APPLICATIONS

This application claims priority to U.S. Application Ser. No. 60/456,920, filed on Mar. 21, 2003, the contents of which is hereby incorporated by reference in its entirety.

### FIELD OF THE INVENTION

The present invention relates to methods and compositions for treating or preventing immunological disorders of the nervous system, e.g., multiple sclerosis, using IL-21/IL-21 receptor agonists, e.g., an IL-21 polypeptide or an active fragment thereof.

### BACKGROUND OF THE INVENTION

Human IL-21 is a cytokine. In its mature form, it is about 131-amino acids in length and has sequence homology to IL-2, IL-4 and IL-15 (Parrish-Novak et al. (2000) Nature 408:57-63). Despite low sequence homology among interleukin cytokines, these cytokines share a common fold that includes a characteristic “four-helix-bundle” structure. Most cytokines bind to either class I or class II cytokine receptors. Class II cytokine receptors include the receptors for IL-10 and the interferons, whereas class I cytokine receptors include the receptors for IL-2, IL-7, IL-9, IL-11-13, and IL-15, as well as the hematopoietic growth factors, leptin and growth hormone (Cosman, D. (1993) Cytokine 5:95-106).

Human IL-21R is a class I cytokine receptor that is expressed by lymphoid cells, particularly by NK, B and T cells (Parrish-Novak et al. (2000) supra). Exemplary nucleic acid sequences encoding human interleukin-21 (IL-21) and its receptor (IL-21R) are described in WO 00/53761, WO 01/85792, Parrish-Novak et al. (2000) supra, and Ozaki et al. (2000) Proc. Natl. Acad. Sci. USA 97:11439-11444, as are the corresponding amino acid sequences. IL-21R shows high sequence homology to IL-2 receptor β chain and IL-4 receptor α chain (Ozaki et al. (2000) supra). Upon ligand binding, IL-21R associates with the common gamma cytokine receptor chain (γc) that is shared by receptors for IL-2, IL-3, IL-4, IL-7, IL-9, IL-13 and IL-15 (Ozaki et al. (2000) supra; Asao et al. (2001) J. Immunol. 167:1-5). The widespread lymphoid distribution of IL-21R suggests that IL-21 may play a role in immune regulation. Indeed, in vitro studies have shown that IL-21 significantly modulates the function of B cells, CD4⁺ and CD8⁺ T cells, and NK cells (Parrish-Novak et al. (2000) supra; Kasaian, M. T. et al. (2002) Immunity. 16:559-569). Parrish-Novak et al. (2000) suggested that IL-21 functions to activate or stimulate the proliferation and maturation of natural killer (NK) cells, the proliferation of mature B-cell populations co-stimulated with anti-CD40, and the proliferation of T cells co-stimulated with anti-CD3.

### SUMMARY OF THE INVENTION

Methods and compositions for increasing the activity of, and/or an interaction between, an interleukin-21 (IL-21) and an IL-21 receptor (also referred to herein as “IL-21R” or “MU-1”) using agonists of IL-21 or IL-21R are disclosed (also referred to herein as an “IL-21/IL-21R agonist” or “agonist”). Such methods and compositions can be used to modulate an immunological disorder of the nervous system or a disease or disorder associated with an IL-10 deficiency. For example, the methods and compositions can be used to treat or prevent multiple sclerosis (MS).

We have shown, for example, that treatment of mice prophylactically with an IL-21/IL-21R agonist, e.g., murine IL-21 polypeptide, results in an amelioration of symptoms in mouse models for experimental autoimmune encephalomyelitis (EAE). The modulation of EAE symptoms was detected in mouse models generated using myelin oligodendrocyte glycoprotein (MOG) peptide (e.g., MOG 35-55) and proteolipid protein (PLP; e.g., PLP 139-151). IL-21 induced proliferation of T cells in vitro. Lymphocytes cultured in the presence of IL-21 produced increased amounts of IL-10 and decreased levels of interferon-γ (IFN-γ). Accordingly, agonists of IL-21/IL-21R activity can be used prophylactically or therapeutically to treat immunological disorders of the nervous system (e.g., chronic immunological disorders of the nervous system, including multiple sclerosis).

Accordingly, in one aspect, the invention features a method of treating (e.g., curing, suppressing, ameliorating, reducing, or delaying) or preventing (e.g., preventing the onset of, or preventing recurrence or relapse of) an immunological disorder of the nervous system (e.g., a chronic immunological disorder of the nervous system, including multiple sclerosis), in a subject. The method includes: administering to the subject an IL-21/IL-21R agonist, in an amount sufficient to modulate immune cell activity and/or number (e.g., to modulate cytokine levels, e.g., cytokine expression, production and/or release), thereby treating or preventing the immunological disorder of the nervous system, e.g., multiple sclerosis. In one embodiment, the IL-21/IL-21R agonist is administered before the onset of symptoms to, e.g., delay or prevent the onset of, or prevent recurrence or relapse of, symptoms. For example, the IL-21/IL-21R agonist can be administered when a subject, e.g., an MS patient, is in remission. In other embodiment, the IL-21/IL-21R agonist is administered after the onset of symptoms or an attack. Exemplary general symptoms of MS include tremor, poor coordination, difficulty walking, and other problems.

The IL-21/IL-21R agonist can be administered to the subject, alone or in combination, with other therapeutic modalities as described herein. Preferably, the subject is a mammal, e.g., a human suffering from an immunological disorder of the nervous system, e.g., multiple sclerosis.

In one embodiment, the IL-21/IL-21R agonist interacts with, e.g., binds to, IL-21 or IL-21R, preferably, mammalian, e.g., human IL-21 or IL-21R, and increases or potentiates one or more IL-21 and/or IL-21R activities. An agonist of IL-21 is referred to herein as an “IL-21 agonist,” and an agonist of IL-21R is referred to as a “IL-21R agonist.” An IL-21 polypeptide is itself an IL-21R agonist. Preferred agonists bind to IL-21 or IL-21R with high affinity, e.g., with an affinity constant of at least about 10⁷ M⁻¹, preferably about 10⁸ M⁻¹, and more preferably, about 10⁹ M−¹ to 10¹⁰ M⁻¹ or stronger. The IL-21/IL-21R agonist can be, e.g., an IL-21 polypeptide or an active fragment thereof, an IL-21 fusion protein, a peptide agonist, an antibody agonist or antigen-binding fragment thereof, or a small molecule agonist.

In one embodiment, the IL-21/IL-21R agonist is an IL-21 polypeptide, e.g., a human, bovine or murine IL-21 polypeptide, or an active fragment thereof (e.g., a human IL-21 polypeptide comprising the amino acid sequence shown as SEQ ID NO:2, or encoded by a nucleotide sequence shown as SEQ ID NO:1, or a sequence at least 85%, 90%, 95%, 98% or more identical thereto). In another embodiment, the IL-21/IL-21R agonist is a murine IL-21 polypeptide or an active fragment thereof (e.g., a murine IL-21 polypeptide comprising the amino acid sequence shown as SEQ ID NO:4, or encoded by a nucleotide sequence shown as SEQ ID NO:3, or a sequence at least 85%, 90%, 95%, 98% or more identical thereto). In yet other embodiments, the IL-21/IL-21R agonist is a fusion protein comprising an IL-21 polypeptide, e.g., human or murine IL-21 polypeptide, or a fragment thereof and, e.g., fused to, a second moiety, e.g., a polypeptide (e.g., a GST, Lex-A, MBP polypeptide sequence or an immunoglobulin chain, including, e.g., an Fc fragment, a heavy chain constant regions of the various isotypes, including: IgG1, IgG2, IgG3, IgG4, IgM, IgA1, IgA2, IgD, and IgE); an agonist antibody or antigen-binding fragment thereof, that binds to the IL-21 receptor; or a small molecule or peptide agonist. In other embodiments, the IL-21/IL-21 R agonist is an agent that increases the activity or levels of IL-21 by, e.g., increasing expression, processing and/or secretion of functional IL-21. Nucleic acids encoding the aforesaid IL-21/IL-21R agonists can also be administered to the subject.

The fusion proteins may additionally include a linker sequence joining the first moiety, e.g., an IL-21 fragment, to the second moiety, e.g., an immunoglobulin fragment. In other embodiments, additional amino acid sequences can be added to the N- or C-terminus of the fusion protein to facilitate expression, steric flexibility, detection and/or isolation or purification.

The IL-21/IL-21R agonists described herein, e.g., IL-21 polypeptide or a fusion protein described herein, can be derivatized or linked to another functional molecule, e.g., another peptide or protein (e.g., an Fab′ fragment). For example, the fusion protein or an antibody, or antigen-binding portion, can be functionally linked (e.g., by chemical coupling, genetic fusion, non-covalent association or otherwise) to one or more other molecular entities, such as an antibody (e.g., a bispecific or a multi-specific antibody), toxins, radioisotopes, cytotoxic or cytostatic agents, among others.

In another embodiment, the IL-21/IL-21R agonist is an antibody, e.g., an agonistic antibody, or antigen-binding fragment thereof, to IL-21R, preferably, human IL-21R. The antibody or antigen-binding fragment thereof, can be a humanized, chimeric, human (e.g., in vitro generated) antibody, or antigen-binding fragment thereof. In one embodiment, the antibody is a bispecific antibody, e.g., an antibody that interacts with IL-21R and another receptor chain.

In one embodiment, the method includes evaluating the subject for an IL-10 parameter. An “IL-10 parameter” is qualitative or quantitative information about IL-10 levels or activity, e.g., IL-10 mRNA or protein levels or activity. The information can include, for example, concentration of IL-10 in one more tissues or one or more samples from a subject. The subject can be evaluated, for example, before the administering, e.g., at least before administering a first dose. The subject can be evaluated, e.g., after the administering, e.g., at after administering one or more doses, e.g., at regular intervals, or in the case of continuous administration, after one or more intervals. The subject can be evaluated both before and after an administration. Information from evaluating an IL-10 parameter can be used to modulate administration of the IL-21/IL-21R agonist. For example, an increase in an IL-10 parameter to a value in the range of normal can indicate that a desired therapeutic effect. A decrease in an IL- 10 parameter can indicate insufficient administration or non-responsiveness. Similarly, it is possible to evaluate a corresponding IFNγ parameter. In such cases, a decrease in an IFNγ parameter to a value in the range of normal can indicate that a desired therapeutic effect. A increase in an IFNγ parameter can indicate insufficient administration or non-responsiveness. Still other parameters that can be evaluated related to other cytokines and factors provided in Table 3.

In one embodiment, the method includes evaluating the subject for a risk of an immunological disorder of the nervous system (e.g., of multiple sclerosis), or one or more symptoms of such a disorder. One method of evaluating risk includes evaluating an IL-10 parameter.

In one implementation, the IL-21/IL-21R agonist is administered in response to an alteration in status of a subject, e.g., in response to a flare-up or attack associated with MS.

The IL-21/IL-21R agonist(s) can be administered in the form of a single dose, or a series of doses separated by intervals of days, weeks or months. The IL-21/IL-21R agonist(s) can be administered by injection, e.g., by injection into the central nervous system, of a subject. For example, the IL-21/IL-21R agonist(s) can be injected into the lumbar cerebrospinal fluid (intrathetically). In other embodiments, the IL-21/IL-21R agonist(s) is administered intravenously.

In one embodiment, the IL-21/IL-21R agonists described herein, e.g., the pharmaceutical compositions thereof, are administered in combination therapy, i.e., combined with other agents, e.g., therapeutic agents, which are useful for treating or preventing an immunological disorder of the nervous system, e.g., multiple sclerosis. For example, the combination therapy can include one or more IL-21/IL-21R agonists, e.g., an IL-21 polypeptide or active fragment thereof, an IL-21 fusion protein, a peptide agonist, an antibody agonist, or a small molecule agonist) co-formulated with, and/or co-administered with, one or more additional therapeutic agents, e.g., one or more cytokine and growth factor inhibitors, immunosuppressants, anti-inflammatory agents, metabolic inhibitors, enzyme inhibitors, and/or cytotoxic or cytostatic agents, as described in more herein.

Examples of therapeutic agents that can be co-administered and/or co-formulated with one or more IL-21/IL-21R agonists to treat multiple sclerosis, include, but are not limited to, one or more of: interferon-β, for example, IFNβ-1α and IFN-β-1 β; a protein that simulates myelin basic protein (e.g., a synthetic protein, e.g., glatiramer acetate, COPAXONE®); corticosteroids; IL-1 inhibitors; TNF inhibitors; antibodies to CD40 ligand and CD80; antagonists of IL-12 and IL-23, e.g., antagonists of a p40 subunit of IL-12 and IL-23 (e.g., inhibitory antibodies against the p40 subunit); IL-22 antagonists; small molecule inhibitors, e.g., methotrexate, leflunomide, sirolimus (rapamycin) and analogs thereof, e.g., CCI-779; Cox-2 and cPLA2 inhibitors; NSAIDs; p38 inhibitors; TPL-2; Mk-2; NFkβ inhibitors; RAGE or soluble RAGE; P-selectin or PSGL-1 inhibitors (e.g., small molecule inhibitors, antibodies thereto, e.g., antibodies to P-selectin); estrogen receptor beta (ERB) agonists or ERB-NFkβ antagonists.

Examples of TNF inhibitors include, e.g., chimeric, humanized, effectively human, human or in vitro generated antibodies, or antigen-binding fragments thereof, that bind to TNF; soluble fragments of a TNF receptor, e.g., p55 or p75 human TNF receptor or derivatives thereof, e.g., 75 kdTNFR-IgG (75 kD TNF receptor-IgG fusion protein, ENBREL™), p55 kD TNF receptor-IgG fusion protein; and TNF enzyme antagonists, e.g., TNFα converting enzyme (TACE) inhibitors.

Additional therapeutic agents that can be co-administered and/or co-formulated with one or more IL-21/IL-21R agonists include one or more of: interferon-β, for example, IFN β-1α and IFN β-1β; COPAXONE®; corticosteroids; IL-1 inhibitors; TNF antagonists (e.g., a soluble fragment of a TNF receptor, e.g., p55 or p75 human TNF receptor or derivatives thereof, e.g., 75 kdTNFR-IgG (75 kD TNF receptor-IgG fusion protein, ENBREL™)); antibodies to CD40 ligand and CD80; and antagonists of IL-12 and/or IL-23, e.g., antagonists of a p40 subunit of IL-12 and IL-23 (e.g., inhibitory antibodies that bind to the p40 subunit of IL-12 and IL-23); methotrexate, leflunomide, and a sirolimus (rapamycin) or an analog thereof, e.g., CCI-779.

In another aspect, the invention features a method for modulating, e.g., increasing or decreasing, immune cell activity and/or number (e.g., the activity and/or number of an immune cell, e.g., a lymphocyte (e.g., a T cell) or a population of immune cells, e.g., a mixed or a substantially purified immune cell population. The method includes contacting an immune cell, e.g., an immune cell as described herein, with an IL-21/IL-21R agonist, e.g., an agonist as described herein, in an amount sufficient to modulate, e.g., increase or decrease, immune cell activity and/or number. In one embodiment, the activity includes modulation, e.g., increase or decrease, of cytokine activity or levels. For example, the IL-21/IL-21R agonist may increase lymphocytic production or levels of IL-10 and/or decrease production or levels of interferon-γ.

The subject method can be used on cells in culture, e.g. in vitro or ex vivo. For example, immune cells, e.g., T cells as described herein, can be cultured in vitro in culture medium and the contacting step can be effected by adding one or more IL-21/IL-21R agonist(s), e.g., an agonist as described herein, to the culture medium. Alternatively, the method can be performed on cells (e.g., immune cells as described herein) present in a subject, e.g., as part of an in vivo (e.g., therapeutic or prophylactic) protocol.

A change in immune cell activity includes any variation(s), e.g., increase/decrease, in one or more of: proliferation, cytokine secretion and/or production, survival, differentiation, cell responsiveness (e.g., desensitization), cytolytic activity, effector cell activity, gene expression, among others, of the immune cell contacted with an IL-21/IL-21R agonist compared to a reference, e.g., an untreated immune cell. For example, contacting an immune cell with an IL-21/IL-21R agonist, e.g., an IL-21 polypeptide, can induce one or more of: proliferation, cytolytic activity, effector cell function, or cytokine secretion of one or more of: thymocytes, lymphocytes, lymph node T cells, mature CD4+ T cells, mature CD8+ T cells, or macrophages. In one embodiment, the IL-21/IL-21R agonist may increase lymphocytic production or levels of IL-10 and/or decrease production or levels of interferon-γ.

In another aspect, the invention features a method of modulating an IL-10 deficiency, or a disorder associated with an IL-10 deficiency in a mammalian subject. The method includes administering to the subject an interleukin-21 (IL-21) polypeptide in an amount sufficient to increase IL-10 expression or activity in the subject, e.g., at least 1.2, 1.5, 2, 2.5, 3, 3.5, 5, or 10 fold increase, e.g., between a 1.2-2.5 fold increase or between a 2.5-5 fold increase, a 5-10 fold increase, or a greater than 10 or 20 fold increase. An “IL-10 deficiency” is a statistically significant decrease in IL-10 relative to a corresponding normal subject. For example, the decrease can have a P value of less than 0.05. Since IL-21 or other IL-21/IL-21R agonists can be used to increase IL-10 levels or activity, IL-21 and such agonists can be used to modulate an IL-10 deficiency.

IL-10 levels can be monitored, for example, in blood, serum, or cerebrospinal fluid. Exemplary disorders that can be associated with an IL-10 deficiency include multiple sclerosis, significant inflammatory events (including ischemia-reperfusion injury), psoriasis and pemphingus. Since IL-21 can increase IL-10 levels, IL-21 can be used to treat at least one symptom of these disorders and others associated with an IL-10 deficiency.

In another aspect, the invention features a method of ameliorating multiple sclerosis in a mammalian subject, e.g., a human. The method includes: administering to the subject an interleukin-21 (IL-21) polypeptide in an amount sufficient to ameliorate multiple sclerosis, or at least one symptom of multiple sclerosis in the subject. For example, where the subject is human, the IL-21 polypeptide can be a human IL-21 polypeptide, e.g., a polypeptide that includes SEQ ID NO:2, or an effectively human IL-21 polypeptide. For example, the polypeptide is recombinantly produced, e.g., in a bacterial cell. In one embodiment, the method includes administering to the subject an interleukin-21 (IL-21) polypeptide in an amount sufficient to increase IL-10 expression or activity in the subject, e.g., at least 1.2, 1.5, 2, 2.5, 3, 3.5, 5, or 10 fold increase, e.g., between a 1.2-2.5 fold increase or between a 2.5-5 fold increase, a 5-10 fold increase, or a greater than 10 or 20 fold increase. The method can include other features described herein.

In another aspect, the invention features a method of treating or preventing an immunological disorder in a mammalian subject. The method includes evaluating an IL-10 parameter in a mammalian subject; and administering, to the subject, an interleukin-21 (IL-21) polypeptide in an amount that is dependent on results of the evaluated IL-10 parameter. For example, the IL-10 parameter includes qualitative or quantitative information about levels of IL-10 protein or IL-10 mRNA. In another example, the IL-10 parameter includes quantitative information about levels of L-10 protein activity.

In one embodiment, the immunological disorder is a neurological disorder. For example, the subject is human and the immunological disorder is multiple sclerosis or a immunological disorder causes damage or alteration to myelin sheaths. The method can include other features described herein.

In another, the invention features a method of evaluating treatment of multiple sclerosis in a mammalian subject. The method includes: administering, to the subject, an agonist of an interleukin-21 (IL-21)/IL-21 receptor (IL-21R) (e.g., an IL-21 polypeptide, an agonistic anti-IL-21R antibody and an antigen-binding fragment of an agonistic anti-IL-21R antibody); and evaluating an IL-10 parameter in the subject. In one embodiment, the method further includes administering to the subject a second dose of the agonist, wherein the second dose is administered as a function of the evaluated IL-10 parameter.

In one embodiment, the subject is human, and the IL-21 polypeptide is a human IL-21 polypeptide, e.g., a polypeptide that includes SEQ ID NO:2.

In one embodiment, the second dose or any subsequent dose is tailored to deliver an interleukin-21 (IL-21) polypeptide in an amount sufficient to increase IL-10 expression or activity in the subject, e.g., at least 1.2, 1.5, 2, 2.5, 3, 3.5, 5, or 10 fold increase, e.g., between a 1.2-2.5 fold increase or between a 2.5-5 fold increase, a 5-10 fold increase, or a greater than 10 or 20 fold increase, e.g., relative to a baseline, e.g., prior to a first treatment. In a related method the first dose is so tailored. The method can include other features described herein.

In another aspect, the invention provides, compositions, e.g., pharmaceutical compositions, which include a pharmaceutically acceptable carrier and at least one of IL-21/IL-21R agonist as described herein (e.g., an IL-21 polypeptide or fusion protein described herein). In one embodiment, the compositions, e.g., pharmaceutical compositions, comprise a combination of two or more one of the aforesaid IL-21/IL-21R agonists. Combinations of the IL-21/IL-21R agonists and a drug, e.g., a therapeutic agent (e.g., one or more cytokine and growth factor inhibitors, immunosuppressants, anti-inflammatory agents, metabolic inhibitors, enzyme inhibitors, and/or cytotoxic or cytostatic agents, as described in more herein) are also within the scope of the invention.

In one embodiment, the pharmaceutical composition includes an IL-21/IL-21R agonist and at least one additional therapeutic agent, in a pharmaceutically-acceptable carrier. Examples of preferred additional therapeutic agents that can be co-formulated in a composition, e.g., a pharmaceutical composition, with one or more IL-21/IL-21R agonists, include, but are not limited to, one or more of: interferon-β, for example, IFNβ-1α and IFNβ-1β; a protein that simulates myelin basic protein (e.g., COPAXONE®); corticosteroids; IL-1 inhibitors; TNF antagonists (e.g., a soluble fragment of a TNF receptor, e.g., p55 or p75 human TNF receptor or derivatives thereof, e.g., 75 kdTNFR-IgG (75 kD TNF receptor-IgG fusion protein, ENBREL™)); antibodies that bind to CD40 ligand and CD80; and antagonists of IL-12 and/or IL-23, e.g., antagonists of a p40 subunit of IL-12 and IL-23 (e.g., inhibitory antibodies against the p40 subunit); methotrexate, leflunomide, and a sirolimus (rapamycin) or an analog thereof, e.g., CCI-779.

In another aspect, the invention features an article of manufacture that includes (i) a container with one or more unit doses of a pharmaceutical composition comprising an IL-21 polypeptide; and (ii) instructions for administering the unit doses to a subject that has, or is suspected of having, multiple sclerosis. For example, the instructions are provided on a label. The label can be affixed to an external surface of the container. In one embodiment, the article further includes a second container, e.g., containing an additional unit dose of a pharmaceutical composition comprising an IL-21 polypeptide. In one embodiment, the article further includes a second container that includes a second pharmaceutical composition comprising an agent for treating multiple sclerosis, i.e., an agent other than IL-21. For example, the agent is glatiramer acetate or another agent described herein. In another embodiment, the pharmaceutical composition that includes an IL-21 polypeptide, in the first container, further includes a second agent agent for treating multiple sclerosis, e.g., glatiramer acetate.

The terms “MU-1” and “IL-21R” are used interchangeably herein. The terms “peptides,” “polypeptides,” and “proteins” are used interchangeably herein. A protein can include one or more chains.

Statistical significance can be determined by any art known method. Exemplary statistical tests include: the Students T-test, Mann Whitney U non-parametric test, and Wilcoxon non-parametric statistical test. Some statistically significant relationships have a P value of less than 0.05 or 0.02. Particular effects mediated by an IL-21/IL-21R agonist may show a difference that is statistically significant (e.g., P value<0.05 or 0.02). The terms “induce”, “inhibit”, “potentiate”, “elevate”, “increase”, “decrease” or the like, e.g., which denote distinguishable qualitative or quantitative differences between two states, and may refer to a difference, e.g., a statistically significant difference, between the two states.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. U.S. Application Ser. No. 60/456,920, filed on Mar. 21, 2003, and PCT patent application Ser. No. XXXXXX, filed on Mar. 22, 2004 in the U.S. Receiving Ofice, attorney docket number 16158-013WO1, titled METHODS AND COMPOSITIONS FOR TREATING IMMUNOLOGICAL DISORDERS USING AGONISTS OF INTERLEUKIN-21/INTERLEUKIN-21 RECEPTOR, and US 2003-0108549 are hereby incorporated by reference in their entireties. In the case of conflict, the present specification, including definitions, controls In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1A is a linear graph depicting the increased proliferation of lymph node cells cultured in the presence of 20 ng/ml of murine IL-21 at various dilutions. For example, at a 1:50 dilution, IL-21 caused increased proliferation of lymph node cells as compared to cells stimulated with peptide alone.

FIG. 1B is a linear graph depicting the increased proliferation of T cells from proteolipid protein (PLP) transgenic mice cultured at the indicated concentration of murine IL-21 (ng/ml) and 1 μg/ml of PLP compared to cells treated with only 1 μg/ml of PLP. The graph shows that IL-21 induces proliferation of T cells from PLP transgenic mice.

FIG. 2 is a bar graph depicting the increased secretion of IL-10 in the presence of the indicated concentrations of mouse IL-21 as compared to untreated cells. The response was saturable and at the highest tested concentration of IL-21 (25 ng/ml), cells produced approximately 2.5-fold more IL-10 than the control group. Production of increased levels of IL-10 suggests that IL-21 treatment can skew antigen-specific cells towards a Th2 profile.

FIG. 3 is a bar graph showing a decrease in secretion of IFNγ by spleen cells and lymph node cells treated with the indicated concentrations of murine IL-21 compared to control cells. Addition of IL-21 to MOG 33-55-stimulated spleen cells from immunized mice results in a two-fold decrease of IFNγ, whereas addition of IL-21R causes a two-fold increase.

FIG. 4 depicts a reduction in clinical symptoms of disease in an EAE model. The figure shows that IL-21 suppresses IFNγ and enhances IL-10. FIG. 4 is a linear graph showing the decrease in EAE symptoms detected in IL-21-treated PLP spleen cells compared to untreated cultures with respect to the days post in vivo transfer.

FIG. 5 depicts a reduction in clinical symptoms of disease in an EAE model. It is a bar graph depicting a decrease in the severity of EAE in mice treated with either a low (100 ng/day) or high (1 μg/day) dose of murine IL-21 compared to control mice.

### DETAILED DESCRIPTION OF THE INVENTION

Methods and compositions for modulating interleukin-21 (IL-21)/IL-21 receptor (MU-1) activity using agonists of IL-21 or IL-21 receptor (“IL-21R” or “MU-1”), are disclosed. In one embodiment, Applicants have shown that treatment of mice with an IL-21/IL-21R agonist, e.g., murine IL-21 polypeptide, results in an amelioration of symptoms in mouse models for experimental autoimmune encephalomyelitis (EAE). The modulation of EAE symptoms was detected in mouse models generated using myelin oligodendrocyte glycoprotein (MOG) peptide (e.g., MOG 35-55) and proteolipid protein (PLP). IL-21 induced proliferation of T cells in vitro. Lymphocytes cultured in the presence of IL-21 produced increased amounts of IL-10 and decreased levels of interferon-γ. Accordingly, agonists of IL-21/IL-21R activity can be used to treat or prevent immunological disorders of the nervous system (e.g., chronic immunological disorders of the nervous system, including multiple sclerosis).

In order that the present invention may be more readily understood, certain terms are first defined. Additional definitions are set forth throughout the detailed description.

The terms “interleukin-21”, “IL-21” and “IL-21 polypeptide” refer to a protein (e.g., a mammalian, e.g., murine or human protein) which is capable of interacting with, e.g., binding to, IL-21R (e.g., a mammalian, e.g., murine or human protein) and having one of the following features: (i) an amino acid sequence of a naturally occurring mammalian IL-21 or a fragment thereof, e.g., an amino acid sequence shown as SEQ ID NO:2 (human) or SEQ ID NO:4 (murine) or a fragment thereof; (ii) an amino acid sequence substantially homologous to, e.g., at least 85%, 90%, 95%, 98%, 99% homologous to, an amino acid sequence shown as SEQ ID NO:2 (human) or SEQ ID NO:4 (murine) or a fragment thereof; (iii) an amino acid sequence which is encoded by a naturally occurring mammalian IL-21 nucleotide sequence or a fragment thereof (e.g., SEQ ID NO:1 (human) or SEQ ID NO:3 (murine), or a fragment thereof, e.g., a region encoding a mature form); (iv) an amino acid sequence encoded by a nucleotide sequence which is substantially homologous to, e.g., at least 85%, 90%, 95%, 98%, 99% homologous to, a nucleotide sequence shown as SEQ ID NO:1 (human) or SEQ ID NO:3 (murine), or a fragment thereof (e.g., a region encoding a mature form); (v) an amino acid sequence encoded by a nucleotide sequence degenerate to a naturally occurring IL-21 nucleotide sequence or a fragment thereof, e.g., SEQ ID NO:1 (human) or SEQ ID NO:3 (murine), or a fragment thereof (e.g., a region encoding a mature form); or (vi) an amino acid sequence, of at least 115 amino acids that is encoded by a nucleotide sequence that hybridizes to the complement of one of the foregoing nucleotide sequences under stringent conditions, e.g., highly stringent conditions (for example, the nucleotide sequence hybridizes in a region that encodes a mature IL-21 protein. IL-21 binding to IL-21R can lead to STAT5 or STAT3 signaling (Ozaki et al. (2000) supra). IL-21 polypeptide can be processed from a nascent protein that includes a signal sequence to a mature protein, from which the signal sequence has been removed.

An “effectively human” IL-21 polypeptide is an IL-21 polypeptide that includes a sufficient number of human amino acid positions such that the polypeptide does not elicit an immunogenic response in a normal human and so that the IL-21 polypeptide interacts with a human IL-21R.

The terms “MU-1,” “MU-1 protein,” “interleukin-21 receptor” or “IL-21R,” refer to a receptor (e.g., of mammalian, e.g., murine or human origin) which is capable of interacting with, e.g., binding to, IL-21 (e.g., of mammalian, e.g., murine or human IL-21) and having one of the following features: (i) an amino acid sequence of a naturally occurring mammalian MU-1 polypeptide IL-21R/MU-1 or a fragment thereof, e.g., an amino acid sequence shown as SEQ ID NO:6 (human) or SEQ ID NO:8 (murine) or a fragment thereof (e.g., the mature region); (ii) an amino acid sequence substantially homologous to, e.g., at least 85%, 90%, 95%, 98%, 99% homologous to, an amino acid sequence shown as SEQ ID NO:6 (human) or SEQ ID NO:8 (murine) or a fragment thereof (e.g., the mature region); (iii) an amino acid sequence which is encoded by a naturally occurring mammalian IL-21R/MU-1 nucleotide sequence (e.g., SEQ ID NO:5 (human) or SEQ ID NO:7 (murine)) or a fragment thereof (e.g., the mature region); (iv) an amino acid sequence encoded by a nucleotide sequence which is substantially homologous to, e.g., at least 85%, 90%, 95%, 98%, 99% homologous to, a nucleotide sequence shown as SEQ ID NO:5 (human) or SEQ ID NO:7 (murine) or a fragment thereof (e.g., the mature region); (v) an amino acid sequence encoded by a nucleotide sequence degenerate to a naturally occurring IL-21R/MU-1 nucleotide sequence or a fragment thereof, e.g., SEQ ID NO:5 (human) or SEQ ID NO:7 (murine) or a fragment thereof (e.g., the mature region); or (vi) an amino acid sequence, of at least 450 amino acids that is encoded a nucleotide sequence that hybridizes to one of the foregoing nucleotide sequence sequences under stringent conditions, e.g., highly stringent conditions. The mature region of the human IL-21R listed in SEQ ID NO:6 is from about amino acids 20-538.

An exemplary IL-21R/MU-1 cDNA was deposited with the American Type Culture Collection on Mar. 10, 1998, as accession number ATCC 98687.

IL-21R is a class I cytokine family receptor, also known as NILR (WO 01/85792; Parrish-Novak et al. (2000) Nature 408:57-63; Ozaki et al. (2000) Proc. Natl. Acad. Sci. USA 97:11439-11444). IL-21R is homologous to the shared β chain of the IL-2 and IL-15 receptors, and IL-4 receptor α chain (Ozaki et al. (2000) supra). Upon ligand binding, IL-21R/MU-1 is capable of interacting with a common γ cytokine receptor chain (γc) (Asao et al. (2001) J. Immunol. 167:1-5), and inducing the phosphorylation of STAT1 and STAT3 (Asao et al. (2001) or STAT5 (Ozaki et al. (2000). IL-21R shows widespread lymphoid tissue distribution.

Forms of IL-21 proteins of less than full length can be used in the methods and compositions, described herein, provided that it retains the ability to bind to an IL-21R polypeptide. IL-21 proteins of less than full length can be produced by expressing a corresponding fragment of the polynucleotide encoding the full-length IL-21 protein in a host cell, or by expressing a polynucleotide encoding a modified protein (e.g., if one or more internal amino acids are removed). One form of IL-21 polypeptide that is less than full length is mature IL-21, e.g., an IL-21 of SEQ ID NO:2. Another form is a polypeptide that is shorter than a full-length, mature IL-21, e.g., less than 131, 130, 129, 128, or 125 amino acids, e.g., between 115 and 130 amino acids in length. For example, an IL-21 polypeptide derived from SEQ ID NO:2 can be missing the final eight amino acids, or a subset thereof, e.g., the IL-21 polypeptide comprises amino acids 1-122. The corresponding polynucleotide fragments can also be used in the methods and compositions of the present invention. Modified polynucleotides as described above may be made by standard molecular biology techniques, including construction of appropriate desired deletion mutants, site-directed mutagenesis methods or by the polymerase chain reaction using appropriate oligonucleotide primers.

The phrase “a biological activity of” a MU-1 or IL-21R polypeptide refers to one or more of the biological activities of the corresponding mature MU-1 protein, including, but not limited to, (1) interacting with, e.g., binding to, an IL-21 polypeptide (e.g., a human IL-21 polypeptide); (2) associating with signal transduction molecules, e.g., γc, jak1; (3) stimulating phosphorylation and/or activation of stat proteins, e.g., STAT5 and/or STAT3; and/or (4) modulating, e.g., stimulating or decreasing, proliferation, differentiation, effector cell function, cytolytic activity, cytokine secretion, and/or survival of immune cells, e.g., T cells (CD8+, CD4+ T cells), NK cells, B cells, macrophages and megakaryocytes).

As used herein, a “IL-21/IL-21R agonist” refers to an agent which potentiates, induces or otherwise enhances one or biological activities of an IL-21R/MU-1 polypeptide, e.g., a biological activity as described herein. For example, the agonist interacts with, e.g., binds to, an IL-21R/MU-1 polypeptide. In one embodiment, an agonist interacts with IL-21R and another receptor chain, e.g., the γ cytokine receptor chain. For example, the agonist crosslinks IL-21R and γ cytokine receptor chain.

As used herein, a “therapeutically effective amount” of an IL-21/IL-21R agonist refers to an amount of an agent which is effective, upon single or multiple dose administration to a subject, e.g., a human patient, at curing, reducing the severity of, ameliorating one or more symptoms of a disorder, e.g., a disorder as described herein, or in prolonging the survival of the subject beyond that expected in the absence of such treatment.

As used herein, “a prophylactically effective amount” of an IL-21/IL-21R agonist refers to an amount of an IL-21/IL-21R agonist which is effective, upon single- or multiple-dose administration to a subject, e.g., a human patient, in preventing or delaying the occurrence of the onset or recurrence of a disorder, e.g., a disorder as described herein.

The term “induce”, “inhibit”, “potentiate”, “elevate”, “increase”, “decrease” or the like, e.g., which denote quantitative differences between two states, refer to at least statistically significant differences between the two states.

The term “in combination” in this context means that the agents are given substantially contemporaneously, either simultaneously or sequentially. If given sequentially, at the onset of administration of the second compound, the first of the two compounds is preferably still detectable at effective concentrations at the site of treatment.

As used herein, a “fusion protein” refers to a protein containing two or more operably associated, e.g., linked, moieties, e.g., protein moieties. Preferably, the moieties are covalently associated. The moieties can be directly associated, or connected via a spacer or linker.

Sequences similar or homologous (e.g., at least about 85% sequence identity) to the sequences disclosed herein are also part of this application. In some embodiment, the sequence identity can be about 90%, 91%, 92%, 93%, 94%, 95%, 96%, 97%, 98%, 99% or higher. Alternatively, substantial identity exists when the nucleic acid segments will hybridize under selective hybridization conditions (e.g., highly stringent hybridization conditions), to the complement of the strand. The nucleic acids may be present in whole cells, in a cell lysate, or in a partially purified or substantially pure form.

Calculations of “homology” or “sequence identity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. The comparison uses the GAP program from the GCG software package (www.gcg.com) and parameters that include a Blossum 62 scoring matrix with a gap penalty of 12, a gap extend penalty of 4, and a frameshift gap penalty of 5.

As used herein, the term “hybridizes under stringent conditions” describes conditions for hybridization and washing. Stringent conditions are known to those skilled in the art and can be found in Current Protocols in Molecular Biology, John Wiley & Sons, N.Y. (1989), 6.3.1-6.3.6. Aqueous and nonaqueous methods are described in that reference and either can be used. A preferred, example of stringent hybridization conditions are hybridization in 6× sodium chloride/sodium citrate (SSC) at about 45° C., followed by one or more washes in 0.2× SSC, 0.1% SDS at 50° C. Another example of stringent hybridization conditions are hybridization in 6× SSC at about 45° C., followed by one or more washes in 0.2× SSC, 0.1% SDS at 55° C. A further example of stringent hybridization conditions are hybridization in 6× SSC at about 45° C., followed by one or more washes in 0.2× SSC, 0.1% SDS at 60° C. Preferably, stringent hybridization conditions are hybridization in 6× SSC at about 45° C., followed by one or more washes in 0.2× SSC, 0.1% SDS at 65° C. Particularly preferred highly stringent conditions (and the conditions that should be used if the practitioner is uncertain about what conditions should be applied to determine if a molecule is within a hybridization limitation) are 0.5M sodium phosphate, 7% SDS at 65° C., followed by one or more washes at 0.2× SSC, 1% SDS at 65° C.

IL-21/IL-21R agonists may have additional conservative or non-essential amino acid substitutions, which do not have a substantial effect on their functions. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

IL-21/IL-21R Agonists

The IL-21/IL-21R agonists used in the methods and compositions can be an IL-21 polypeptide, e.g., a human or murine IL-21 polypeptide, or an active fragment thereof (e.g., a human IL-21 polypeptide comprising the amino acid sequence shown as SEQ ID NO:2, or an amino acid sequence including a region encoded by a nucleotide sequence shown as SEQ ID NO:1, or a sequence at least 85%, 90%, 95%, 98% or more identical thereto)(e.g., the region of SEQ ID NO:1 that encodes a mature IL-21 polypeptide. In another embodiment, the IL-21/IL-21R agonist is a murine IL-21 polypeptide or an active fragment thereof (e.g., a murine IL-21 polypeptide comprising the amino acid sequence shown as SEQ ID NO:4, or encoded by a nucleotide sequence shown as SEQ ID NO:3, or a sequence at least 85%, 90%, 95%, 98% or more identical thereto), or an IL-21 polypeptide from another mammal, e.g., a non-human primate, a bovine, and so forth.

Amino acid sequences of IL-21 polypeptides are publicly known. For example, the nucleotide sequence and amino acid sequence of a human IL-21 is available at GENBANK® Acc. No. X_{—}011082. An exemplary disclosed human IL-21 nucleotide sequence is presented below:

| (SEQ ID NO:1) | |
|---|---|
| 1 | gctgaagtga aaacgagacc aaggtctagc tctactgttg gtacttatga gatccagtcc |
| 61 | tggcaacatg gagaggattg tcatctgtct gatggtcatc ttcttgggga cactggtcca |
| 121 | caaatcaagc tcccaaggtc aagatcgcca catgattaga atgcgtcaac ttatagatat |
| 181 | tgttgatcag ctgaaaaatt atgtgaatga cttggtccct gaatttctgc cagctccaga |
| 241 | agatgtagag acaaactgtg agtggtcagc tttttcctgc tttcagaagg cccaactaaa |
| 301 | gtcagcaaat acaggaaaca atgaaaggat aatcaatgta tcaattaaaa agctgaagag |
| 361 | gaaaccacct tccacaaatg cagggagaag acagaaacac agactaacat gcccttcatg |
| 421 | tgattcttat gagaaaaaac cacccaaaga attcctagaa agattcaaat cacttctcca |
| 481 | aaagatgatt catcagcatc tgtcctctag aacacacgga agtgaagatt cctgaggatc |
| 541 | taacttgcag ttggacacta tgttacatac tctaatatag tagtgaaagt catttctttg |
| 601 | tattccaagt ggaggag |

Additional nucleotide sequence information is available, e.g., from AF254069 [gi:11093535] which provides a 642 bp mRNA sequence encoding an exemplary IL-21 polypeptide. In some embodiments, it is sufficient to use the region of nucleotide sequence that encodes mature IL-21, e.g., without a region encoding a signal sequence. The amino acid sequence of an exemplary mature human IL-21 polypeptide is presented below:

(SEQ ID NO:2)

QDRHMIRMRQLIDIVDQLKNYVNDLVPEFLPAPEDVETNCEWSAFSCFQK
AQLKSANTGNNERIINVSIKKLKRKPPSTNAGRRQKHRLTCPSCDSYEKK
PPKEFLERFKSLLQKMIHQHLSSRTHGSEDS

The mature sequence is based on Parrish-Novak et al. (2000) Nature 408:57-63. The full length sequence is:

(SEQ ID NO:10)

MRSSPGNMERIVICLMVIFLGTLVHKSSSQGQDRHMIRMRQLIDIVDQLK
NYVNDLVPEFLPAPEDVETNCEWSAFSCFQKAQLKSANTGNNERIINVSI
KKLKRKPPSTNAGRRQKHRLTCPSCDSYEKKPPKEFLERFKSLLQKMIHQ
HLSSRTHGSEDS

Additional entries providing amino acid sequences for human IL-21 polypeptides are as follows: gi|11141875|ref|NP_{—}068575.1| interleukin 21 [Homo sapiens]; gi|11093536|gb|AAG29348.1| interleukin 21 [Homo sapiens]; gi|42542586|gb|AAH66259.1| Interleukin 21 [Homo sapiens]; gi|42542588|gb|AAH66260.1| Interleukin 21 [Homo sapiens]; gi|42542657|gb|AAH66261.1| Interleukin 21 [Homo sapiens]; gi|42542659|gb|AAH66258.1| Interleukin 21 [Homo sapiens]; and gi|42542807|gb|AAH66262.1| Interleukin 21 [Homo sapiens]. The human IL-21 polypeptide can be a variant of a polypeptide described herein, provided that it retains functionality.

IL-21 polypeptides can be encoded by nucleic acids that hybridize to the nucleotide sequence set forth in SEQ ID NO:1, SEQ ID NO:3, SEQ ID NO:5, or SEQ ID NO:7, or the complement thereof, under a condition described herein, e.g., highly stringent conditions (for example, 0.1× SSC at 65° C.). Isolated polynucleotides which encode IL-21/IL-21R proteins or fusion proteins, but which differ from the nucleotide sequence set forth in SEQ ID NO:1, SEQ ID NO:3, SEQ ID NO:5, or SEQ ID NO:7, by virtue of the degeneracy of the genetic code can be used. Variations in the nucleotide sequence as set forth in SEQ ID NO:1, SEQ ID NO:3, SEQ ID NO:5, or SEQ ID NO:7, which are caused by point mutations or by induced modifications can also be used.

In yet other embodiments, the IL-21/IL-21R agonist is a fusion protein comprising an IL-21 polypeptide, e.g., human or murine IL-21 polypeptide, or a fragment thereof and, e.g., fused to, a second moiety, e.g., a polypeptide (e.g., a GST, Lex-A, MBP polypeptide sequence or an immunoglobulin chain, including, e.g., an Fc fragment, a heavy chain constant regions of the various isotypes, including: IgG1, IgG2, IgG3, IgG4, IgM, IgA1, IgA2, IgD, and IgE).

The fusion proteins may additionally include a linker sequence joining the IL-21 or IL-21R fragment to the second moiety. For example, the fusion protein can include a peptide linker, e.g., a peptide linker of about 4 to 20, more preferably, 5 to 10, amino acids in length; the peptide linker is 8 amino acids in length. Each of the amino acids in the peptide linker is selected from the group consisting of Gly, Ser, Asn, Thr and Ala; the peptide linker includes a Gly-Ser element. In other embodiments, the fusion protein includes a peptide linker and the peptide linker includes a sequence having the formula (Ser-Gly-Gly-Gly-Gly, SEQ ID NO:11)_{y} wherein y is, e.g., 1, 2, 3, 4, 5, 6, 7, or 8.

In other embodiments, additional amino acid sequences can be added to the N- or C-terminus of the fusion protein to facilitate expression, detection and/or isolation or purification. For example, IL-21 fusion protein may be linked to one or more additional moieties, e.g., GST, His6 tag, FLAG tag. For example, the fusion protein may additionally be linked to a GST fusion protein in which the fusion protein sequences are fused to the C-terminus of the GST (i.e., glutathione S-transferase) sequences. Such fusion proteins can facilitate the purification of the fusion protein.

In another embodiment, the fusion protein is includes a heterologous signal sequence (i.e., a polypeptide sequence that is not present in a polypeptide encoded by a IL-21 nucleic acid) at its N-terminus. For example, the native signal sequence can be removed and replaced with a signal sequence from another protein. In certain host cells (e.g., mammalian host cells), expression and/or secretion of IL-21/IL-21R agonist can be increased through use of a heterologous signal sequence. IL-21R proteins and fragments thereof can also be produced using similar methods, e.g., to provide an immunogen to obtain agonizing antibodies that interact with IL-21R.

A chimeric or fusion protein can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (eds.) CURRENT PROTOCOLS IN MOLECULAR BIOLOGY, John Wiley & Sons, 1992). Moreover, many expression vectors are commercially available that encode a fusion moiety (e.g., an Fc region of an immunoglobulin heavy chain). An IL-21/IL-21R agonist encoding nucleic acid can be cloned into such an expression vector such that the fusion moiety is linked in-frame to the immunoglobulin protein.

In some embodiments, the fusion polypeptides can exist as oligomers, such as dimers (e.g., homo- or hetero-dimers) or trimers. The first polypeptide, and/or nucleic acids encoding the first polypeptide, can be constructed using methods known in the art.

In some embodiments, the first polypeptide includes full-length IL-21/IL-21R agonist polypeptide (e.g., IL-21 itself). Alternatively, the first polypeptide comprises less than full-length, IL-21/IL-21R polypeptide. A signal peptide that can be included in the fusion protein is MPLLLLLLLLPSPLHP (SEQ ID NO:9). If desired, one or more amino acids can additionally be inserted between the first polypeptide moiety comprising the IL-21/IL-21R agonist moiety and the second polypeptide moiety.

The second polypeptide is preferably soluble. In some embodiments, the second polypeptide enhances the half-life, (e.g., the serum half-life) of the linked polypeptide. In some embodiments, the second polypeptide includes a sequence that facilitates association of the fusion polypeptide with a second IL-21/IL-21R agonist polypeptide. In preferred embodiments, the second polypeptide includes at least a region of an immunoglobulin polypeptide. Immunoglobulin fusion polypeptide are known in the art and are described in e.g., U.S. Pat. Nos. 5,516,964; 5,225,538; 5,428,130; 5,514,582; 5,714,147; and 5,455,165.

In some embodiments, the second polypeptide comprises a full-length immunoglobulin polypeptide. Alternatively, the second polypeptide comprises less than full-length immunoglobulin polypeptide, e.g., a heavy chain, light chain, Fab, Fab₂, Fv, or Fc. The second polypeptide can include the heavy chain of an immunoglobulin polypeptide. The second polypeptide can include the Fc region of an immunoglobulin polypeptide.

In some embodiments, the second polypeptide has less effector function that the effector function of an Fc region of a wild-type immunoglobulin heavy chain. Fc effector function includes for example, Fc receptor binding, complement fixation and T cell depleting activity (see for example, U.S. Pat. No. 6,136,310). Methods for assaying T cell depleting activity, Fc effector function, and antibody stability are known in the art. In one embodiment the second polypeptide has low or no affinity for the Fc receptor. In an alternative embodiment, the second polypeptide has low or no affinity for complement protein C1q.

The isolated IL-21/IL-21R agonist polynucleotides described herein may be operably linked to an expression control sequence such as the pMT2 or pED expression vectors disclosed in Kaufman et al., Nucleic Acids Res. 19, 4485-4490 (1991), in order to produce the protein recombinantly. Many suitable expression control sequences are known in the art. General methods of expressing recombinant proteins are also known and are exemplified in R. Kaufman, Methods in Enzymology 185, 537-566 (1990). As defined herein “operably linked” means enzymatically or chemically ligated to form a covalent bond between a particular polynucleotide encoding a protein of interest and the expression control sequence, in such a way that the protein of interest (e.g., IL-21 or another IL-21/IL-21R agonist) is expressed by a host cell which has been transformed (transfected) with the ligated polynucleotide/expression control sequence.

The term “vector”, as used herein, is intended to refer to a nucleic acid molecule capable of transporting, or sustaining maintenance or replication of, another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “regulatory sequence” is intended to includes promoters, enhancers and other expression control elements (e.g., polyadenylation signals) that control the transcription or translation of the antibody chain genes. Such regulatory sequences are described, for example, in Goeddel; Gene Expression Technology: Methods in Enzymology 185, Academic Press, San Diego, Calif. (1990). It will be appreciated by those skilled in the art that the design of the expression vector, including the selection of regulatory sequences may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from FF-1a promoter and BGH poly A, cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al.

The recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr⁻ host cells with methotrexate selection/amplification) and the neo gene (for G418 selection). A number of types of cells may act as suitable host cells for expression of the IL-21/IL-21R agonist protein or fusion protein thereof. Any cell type capable of expressing functional IL-21/IL-21R protein may be used. Suitable mammalian host cells include, for example, monkey COS cells, Chinese Hamster Ovary (CHO) cells, human kidney 293 cells, human epidermal A431 cells, human Colo205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK, Rat2, BaF3, 32D, FDCP-1, PC12, M1x or C2C12 cells.

The IL-21/IL-21R agonist protein or fusion protein thereof may also be produced by operably linking a polynucleotide encoding such a protein to suitable control sequences in one or more insect expression vectors, and employing an insect expression system. Materials and methods for baculovirus/insect cell expression systems are commercially available in kit form from, e.g., Invitrogen, San Diego, Calif. U.S.A. (the MAXBAC® kit), and such methods are well known in the art, as described in Summers and Smith, Texas Agricultural Experiment Station Bulletin No. 1555 (1987), incorporated herein by reference. Soluble forms of the MU-1 protein may also be produced in insect cells using appropriate isolated polynucleotides as described above.

Alternatively, the IL-21/IL-21R agonist protein or fusion protein thereof may be produced in lower eukaryotes such as yeast or in prokaryotes such as bacteria. Suitable yeast strains include Saccharomyces cerevisiae, Schizosaccharomyces pombe, Kluyveromyces strains, Candida, or any yeast strain capable of expressing heterologous proteins. Suitable bacterial strains include Escherichia coli, Bacillus subtilis, Salmonella typhimurium, or any bacterial strain capable of expressing heterologous proteins.

In one embodiment, IL-21 is produced in a bacterial without a signal sequence (e.g., without either a prokaryotic or eukaryotic signal sequence). Expression in bacteria may result in formation of inclusion bodies incorporating the recombinant protein. Thus, refolding of the recombinant protein may be required in order to produce active or more active material. Several methods for obtaining correctly folded heterologous proteins from bacterial inclusion bodies are known in the art. These methods generally involve solubilizing the protein from the inclusion bodies, then denaturing the protein completely using a chaotropic agent.

When cysteine residues are present in the primary amino acid sequence of the protein, the protein can be refolded in an environment which facilitates correct formation of disulfide bonds (e.g., a redox system). General methods of refolding are disclosed in Kohno, Meth. Enzym., 185:187-195 (1990). EP 0433225 and U.S. Pat. No. 5,399,677. Asano et al. (2002) FEBS Lett. 528(1-3):70-6 describes an exemplary method for refolding IL-21 produced in bacterial cells. For example, rIL-21 (recombinant IL-21) is expressed as insoluble inclusion bodies in E. coli, then solubilized (e.g., using a denaturant) and refolded by using a modified dialysis method in which redox reagents are introduced.

The IL-21/IL-21R agonist protein or fusion protein thereof may also be expressed as a product of transgenic animals, e.g., as a component of the milk of transgenic cows, goats, pigs, or sheep which are characterized by somatic or germ cells containing a polynucleotide sequence encoding the IL-21/IL-21R agonist protein or fusion protein thereof.

The IL-21/IL-21R agonist protein or fusion protein thereof may be prepared by growing a culture transformed host cells under culture conditions necessary to express the desired protein. The resulting expressed protein may then be purified from the culture medium or cell extracts. Soluble forms of the IL-21/IL-21R agonist protein or fusion protein thereof can be purified from conditioned media. Membrane-bound forms of MU-1 protein can be purified by preparing a total membrane fraction from the expressing cell and extracting the membranes with a non-ionic detergent such as Triton X-100.

The IL-21/IL-21R agonist protein or fusion protein can be purified using methods known to those skilled in the art. For example, the IL-21/IL-21R agonist protein can be concentrated using a commercially available protein concentration filter, for example, an AMICON® or MILLIPORE® PELLICON™ ultrafiltration unit. Following the concentration step, the concentrate can be applied to a purification matrix such as a gel filtration medium. Alternatively, an anion exchange resin can be employed, for example, a matrix or substrate having pendant diethylaminoethyl (DEAE) or polyetheyleneimine (PEI) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Sulfopropyl groups are preferred (e.g., S-Sepharose® columns). The purification of the MU-1 protein or fusion protein from culture supernatant may also include one or more column steps over such affinity resins as concanavalin A-agarose, heparin-TOYOPEARL® or Cibacrom blue 3GA SEPHAROSE®; or by hydrophobic interaction chromatography using such resins as phenyl ether, butyl ether, or propyl ether; or by immunoaffinity chromatography. Finally, one or more reverse-phase high performance liquid chromatography (RP-HPLC) steps employing hydrophobic RP-HPLC media, e.g., silica gel having pendant methyl or other aliphatic groups, can be employed to further purify the IL-21/IL-21R agonist protein. Affinity columns including antibodies to the IL-21/IL-21R agonist protein can also be used in purification in accordance with known methods. Some or all of the foregoing purification steps, in various combinations or with other known methods, can also be employed to provide a substantially purified isolated recombinant protein. Preferably, the isolated IL-21/IL-21R agonist protein is purified so that it is substantially free of other mammalian proteins or, if produced in bacteria, substantially free of other bacterial proteins, e.g., endotoxins.

In other embodiments, the IL-21/IL-21R agonists are antibodies, or antigen-binding fragments thereof, that bind to, e.g., IL-21R, preferably, mammalian (e.g., human or murine) IL-21 or IL-21R, and activate an IL-21R activity.

As used herein, the term “antibody” refers to a protein comprising at least one, and preferably two, heavy (H) chain variable regions (abbreviated herein as VH), and at least one and preferably two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, which are incorporated herein by reference). Each VH and VL is composed of three CDR's and four FRs, arranged from amino-terminus to carboxy-terminus in the following order: FR1, CDR1, FR2, CDR2, FR3, CDR3, FR4.

The antibody can further include a heavy and light chain constant region, to thereby form a heavy and light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region is comprised of three domains, CH1, CH2 and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system.

As used herein, the term “immunoglobulin” refers to a protein consisting of one or more polypeptides substantially encoded by immunoglobulin genes. The recognized human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 Kd or 214 amino acids) are encoded by a variable region gene at the NH2-terminus (about 110 amino acids) and a kappa or lambda constant region gene at the COOH-terminus. Full-length immunoglobulin “heavy chains” (about 50 Kd or 446 amino acids), are similarly encoded by a variable region gene (about 116 amino acids) and one of the other aforementioned constant region genes, e.g., gamma (encoding about 330 amino acids).

As used herein, “isotype” refers to the antibody class (e.g., IgM or IgG1) that is encoded by heavy chain constant region genes.

The term “antigen-binding fragment” of an antibody (or simply “antibody portion,” or “fragment”), as used herein, refers to one or more fragments of a full-length antibody that retain the ability to specifically bind to an antigen (e.g., IL-21R). Examples of binding fragments encompassed within the term “antigen-binding fragment” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. These antibody fragments are obtained using conventional techniques known to those with skill in the art, and the fragments are screened for utility in the same manner as are intact antibodies. An “effectively human” immunoglobulin variable region is an immunoglobulin variable region that includes a sufficient number of human framework amino acid positions such that the immunoglobulin variable region does not elicit an immunogenic response in a normal human. An “effectively human” antibody is an antibody that includes a sufficient number of human amino acid positions such that the antibody does not elicit an immunogenic response in a normal human. Human and effectively human immunoglobulin variable regions and antibodies can be used.

IL-21 or IL-21R proteins may be used to immunize animals (e.g., non-human animals and non-human animals include human immunoglobulin genes) to obtain polyclonal and monoclonal antibodies which specifically react with the IL-21/IL-21R agonist protein and which may activate an IL-21R. Such antibodies may be obtained using the entire IL-21/IL-21R agonist protein as an immunogen, or by using fragments of IL-21/IL-21R. The peptide immunogens additionally may contain a cysteine residue at the carboxyl terminus, and are conjugated to a hapten such as keyhole limpet hemocyanin (KLH). Additional peptide immunogens may be generated by replacing tyrosine residues with sulfated tyrosine residues. Methods for synthesizing such peptides are known in the art, for example, as in R. P. Merrifield, J. Amer. Chem. Soc. 85, 2149-2154 (1963); J. L. Krstenansky, et al., FEBS Lett. 211, 10 (1987).

Human monoclonal antibodies (mAbs) directed against IL-21 or IL-21R can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., WO 91/00906, WO 91/10741; WO 92/03918; WO 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

Monoclonal antibodies can also be generated by other methods known to those skilled in the art of recombinant DNA technology. An alternative method, referred to as the “combinatorial antibody display” method, has been developed to identify and isolate antibody fragments having a particular antigen specificity, and can be utilized to produce monoclonal antibodies (for descriptions of combinatorial antibody display see e.g., Sastry et al. 1989 PNAS 86:5728; Huse et al. 1989 Science 246:1275; and Orlandi et al. 1989 PNAS 86:3833). After immunizing an animal with an immunogen as described above, the antibody repertoire of the resulting B-cell pool is cloned. Methods are generally known for obtaining the DNA sequence of the variable regions of a diverse population of immunoglobulin molecules by using a mixture of oligomer primers and PCR. For instance, mixed oligonucleotide primers corresponding to the 5′ leader (signal peptide) sequences and/or framework 1 (FR1) sequences, as well as primer to a conserved 3′ constant region primer can be used for PCR amplification of the heavy and light chain variable regions from a number of murine antibodies (Larrick et al., 1991, Biotechniques 11:152-156). A similar strategy can also been used to amplify human heavy and light chain variable regions from human antibodies (Larrick et al., 1991, Methods: Companion to Methods in Enzymology 2:106-110).

Chimeric antibodies, including chimeric immunoglobulin chains, can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

An antibody or an immunoglobulin chain can be humanized by methods known in the art. Humanized antibodies, including humanized immunoglobulin chains, can be generated by replacing sequences of the Fv variable region which are not directly involved in antigen binding with equivalent sequences from human Fv variable regions. General methods for generating humanized antibodies are provided by Morrison, S. L., 1985, Science 229:1202-1207, by Oi et al., 1986, BioTechniques 4:214, and by Queen et al. U.S. Pat. No. 5,585,089, U.S. Pat. No. 5,693,761 and U.S. Pat. No. 5,693,762, the contents of all of which are hereby incorporated by reference. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a predetermined target. The recombinant DNA encoding the humanized antibody, or fragment thereof, can then be cloned into an appropriate expression vector.

Humanized or CDR-grafted antibody molecules or immunoglobulins can be produced by CDR-grafting or CDR substitution, wherein one, two, or all CDR's of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies (UK Patent Application GB 2188638A, filed on Mar. 26, 1987; Winter U.S. Pat. No. 5,225,539), the contents of which is expressly incorporated by reference. All of the CDR's of a particular human antibody may be replaced with at least a portion of a non-human CDR or only some of the CDR's may be replaced with non-human CDR's. It is only necessary to replace the number of CDR's required for binding of the humanized antibody to a predetermined antigen.

In some implementations, monoclonal, chimeric and humanized antibodies can be modified by, e.g., deleting, adding, or substituting other portions of the antibody, e.g., the constant region. For example, an antibody can be modified as follows: (i) by deleting the constant region; (ii) by replacing the constant region with another constant region, e.g., a constant region meant to increase half-life, stability or affinity of the antibody, or a constant region from another species or antibody class; or (iii) by modifying one or more amino acids in the constant region to alter, for example, the number of glycosylation sites, effector cell function, Fc receptor (FcR) binding, complement fixation, among others.

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. No. 5,624,821 and U.S. Pat. No. 5,648,260). Similar type of alterations could be described which if applied to the murine, or other species immunoglobulin would reduce or eliminate these functions.

For example, it is possible to alter the affinity of an Fc region of an antibody (e.g., an IgG, such as a human IgG) for an FcR (e.g., Fc gamma R1), or for C1q binding by replacing the specified residue(s) with a residue(s) having an appropriate functionality on its side chain, or by introducing a charged functional group, such as glutamate or aspartate, or perhaps an aromatic non-polar residue such as phenylalanine, tyrosine, tryptophan or alanine (see e.g., U.S. Pat. No. 5,624,821).

In one embodiment, an agonist of IL-21R is an agent that interacts with IL-21R and another receptor subunit, e.g., γc. For example, the agent can be a protein that interacts with IL-21R and another receptor subunit, e.g., γc. The protein can be, e.g., a bispecific antibody that includes one antigen binding site that interacts with IL-21R and another antigen binding site that interacts with γc. Binding of such a protein can be used to crosslink and agonize the receptor, e.g., activate or increase STAT3 or STAT5 signalling.

In one embodiment, an IL-21/IL-21R agonist is an agent (e.g., an immunoglobulin) that stabilizes an IL-21/IL-21R interaction, e.g., by binding one or both of IL-21 and IL-21R.

Agonists of IL-21/IL-21R proteins can be screened for, e.g., binding and/or activation of an IL-21R polypeptide using procedures known in the art. Binding assays using a desired binding protein, immobilized or not, are known in the art and may be used for this purpose using the IL-21R protein as described herein. Purified cell based or protein based (cell free) screening assays may be used to identify such agonists. For example, IL-21R protein may be immobilized in purified form on a carrier and binding or potential ligands to purified IL-21R protein may be measured. Cell-based assays for evaluating IL-21R activity and STAT3 or STAT5 signalling are known. Examples are described herein.

Pharmaceutical Compositions

IL-21/IL-21R-agonists may be used as a pharmaceutical composition when combined with a pharmaceutically acceptable carrier. Such a composition may contain, in addition to the IL-21/IL-21R-agonists and carrier, various diluents, fillers, salts, buffers, stabilizers, solubilizers, and other materials well known in the art. The term “pharmaceutically acceptable” means a non-toxic material that does not interfere with the effectiveness of the biological activity of the active ingredient(s). The characteristics of the carrier will depend on the route of administration.

The pharmaceutical composition may further contain other anti-inflammatory agents as described in more detail below. Such additional factors and/or agents may be included in the pharmaceutical composition to produce a synergistic effect with an IL-21/IL-21R-agonists, or to minimize side effects caused by the IL-21/IL-21R-agonists. Conversely IL-21/IL-21R-agonists may be included in formulations of the particular anti-inflammatory agent to minimize side effects of the anti-inflammatory agent.

The pharmaceutical composition may be in the form of a liposome in which IL-21/IL-21R-agonists is combined, in addition to other pharmaceutically acceptable carriers, with amphipathic agents such as lipids which exist in aggregated form as micelles, insoluble monolayers, liquid crystals, or lamellar layers which in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. No. 4,235,871; U.S. Pat. No. 4,501,728; U.S. Pat. No. 4,837,028; and U.S. Pat. No. 4,737,323, all of which are incorporated herein by reference.

As used herein, the term “therapeutically effective amount” means the total amount of each active component of the pharmaceutical composition or method that is sufficient to show a meaningful patient benefit, e.g., amelioration of symptoms of, healing of, or increase in rate of healing of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

In practicing the method of treatment or use, a therapeutically effective amount of an IL-21/IL-21R-agonist is administered to a subject, e.g., mammal (e.g., a human). An IL-21/IL-21R-agonists may be administered either alone or in combination with other therapies such as treatments employing anti-inflammatory agents. When co-administered with one or more agents, an IL-21- and/or IL-21R-agonist may be administered either simultaneously with the second agent, or sequentially. If administered sequentially, the attending physician can decide on the appropriate sequence of administering an IL-21/IL-21R-agonist in combination with other agents.

Administration of an IL-21/IL-21R-agonist used in the pharmaceutical composition or to practice the method of the present invention can be carried out in a variety of conventional ways, such as oral ingestion, intracranial, inhalation, or cutaneous, subcutaneous, or intravenous injection or administration. For example, the composition can be delivered as an epidural or otherwise, e.g., to cerebrospinal fluid.

When a therapeutically effective amount of an IL-21/IL-21R-agonist is administered orally, the binding agent will be in the form of a tablet, capsule, powder, solution or elixir. When administered in tablet form, the pharmaceutical composition may additionally contain a solid carrier such as a gelatin or an adjuvant. The tablet, capsule, and powder contain from about 5 to 95% binding agent, and preferably from about 25 to 90% binding agent. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of the binding agent, and preferably from about 1 to 50% the binding agent.

When a therapeutically effective amount of an IL-21/IL-21R-agonist is administered by intravenous, cutaneous or subcutaneous injection, binding agent will be in the form of a pyrogen-free, parenterally acceptable aqueous solution. The preparation of such parenterally acceptable protein solutions, having due regard to pH, isotonicity, stability, and the like, is within the skill in the art. A preferred pharmaceutical composition for intravenous, cutaneous, or subcutaneous injection should contain, in addition to binding agent an isotonic vehicle such as sodium chloride injection, Ringer's injection, dextrose injection, dextrose and sodium chloride injection, lactated Ringer's injection, or other vehicle as known in the art. The pharmaceutical composition of the present invention may also contain stabilizers, preservatives, buffers, antioxidants, or other additive known to those of skill in the art.

The amount of an IL-21/IL-21R-agonist in the pharmaceutical composition of the present invention can depend upon the nature and severity of the condition being treated, and on the nature of prior treatments that the patient has undergone. The attending physician can decide the amount of agonist with which to treat each individual patient. Initially, for example, the attending physician can administer low doses of binding agent and observe the patient's response. Larger doses of binding agent may be administered until the optimal therapeutic effect is obtained for the patient, and at that point the dosage is not generally increased further, or by monitoring cytokine levels or one or more symptoms. It is contemplated that the various pharmaceutical compositions used to practice the method of the present invention should contain about 0.1 μg to about 100 mg IL-21/IL-21R-agonist per kg body weight. For example, useful dosages can include between about 10 μg-1 mg, 0.1-5 mg, and 3-50 mg of IL-21/IL-21R agonist per kg body weight. Useful dosages of IL-21 can further include between about 5 μg-1 mg, 0.1-5 mg, and 3-20 mg of IL-21/IL-21R agonist per kg body weight.

The duration of intravenous therapy using the pharmaceutical composition can vary, depending on the severity of the disease being treated and the condition and potential idiosyncratic response of each individual patient. The duration of each application of the IL-21/IL-21R-agonist can be, e.g., in the range of 12 to 24 hours of continuous intravenous administration. The attending physician can decide on the appropriate duration of intravenous therapy using the pharmaceutical composition of the present invention.

In addition to IL-21/IL-21 agonists, where such agonists are proteins, the disease or disorder can be treated or prevented by administration or use of polynucleotides encoding such proteins (such as, for example, in gene therapies or vectors suitable for introduction of DNA).

Uses of IL-21/IL-21R-Agonists to Enhance an Immune Response

In one aspect, the present invention provides methods for treating (e.g., curing, suppressing, ameliorating, delaying or preventing the onset of, or preventing recurrence or relapse of) or preventing an immunological disorder of the nervous system (e.g., a chronic immunological disorder of the nervous system, including multiple sclerosis), in a subject. The method includes: administering to the subject an IL-21/IL-21R agonist, in an amount sufficient to modulate immune cell activity and/or cell number (e.g., to modulate cytokine levels, e.g., cytokine expression, production and/or release), thereby treating or preventing the immunological disorder of the nervous system, e.g., multiple sclerosis.

Multiple sclerosis (MS) is a central nervous system disease that is characterized by inflammation and loss of myelin sheaths—the fatty material that insulates nerves and is needed for proper nerve function. Inflammation that results from an immune response that is modulated by on IL-21 can be prevented or treated with the IL-21/IL-21R agonists described herein. In the experimental autoimmune encephalitis (EAE) mouse model for multiple sclerosis (Tuohy et al. (J. Immunol. (1988) 141: 1126-1130), Sobel et al. (J. Immunol. (1984) 132: 2393-2401), and Traugott (Cell Immunol. (1989) 119: 114-129), treatment of mice with IL-21 injections prior to EAE induction reduces the symptoms of the disease. Accordingly, the IL-21/IL-21R agonists described herein may similarly be used to treat or prevent multiple sclerosis in humans.

Patients suitable for such treatment may be identified by criteria establishing a diagnosis of clinically definite MS as defined by the workshop on the diagnosis of MS (Poser et al., Ann. Neurol. 13:227, 1983). Briefly, an individual with clinically definite MS has had two attacks and clinical evidence of either two lesions or clinical evidence of one lesion and paraclinical evidence of another, separate lesion. Definite MS may also be diagnosed by evidence of two attacks and oligoclonal bands of IgG in cerebrospinal fluid or by combination of an attack, clinical evidence of two lesions and oligoclonal band of IgG in cerebrospinal fluid. Slightly lower criteria are used for a diagnosis of clinically probable MS.

Effective treatment of multiple sclerosis may be examined in several different ways. Satisfying any of the following criteria evidences effective treatment. Three main criteria are used: EDSS (extended disability status scale), appearance of exacerbations or MRI (magnetic resonance imaging). The EDSS is a means to grade clinical impairment due to MS (Kurtzke, Neurology 33:1444, 1983). Eight functional systems are evaluated for the type and severity of neurologic impairment. Briefly, prior to treatment, patients are evaluated for impairment in the following systems: pyramidal, cerebella, brainstem, sensory, bowel and bladder, visual, cerebral, and other. Follow-ups are conducted at defined intervals. The scale ranges from 0 (normal) to 10 (death due to MS). A decrease of one full step defines an effective treatment in the context of the present invention (Kurtzke, Ann. Neurol. 36:573-79, 1994). Exacerbations are defined as the appearance of a new symptom that is attributable to MS and accompanied by an appropriate new neurologic abnormality (IFNB MS Study Group, supra). In addition, the exacerbation must last at least 24 hours and be preceded by stability or improvement for at least 30 days. Briefly, patients are given a standard neurological examination by clinicians. Exacerbations are either mild, moderate, or severe according to changes in a Neurological Rating Scale (Sipe et al., Neurology 34:1368, 1984). An annual exacerbation rate and proportion of exacerbation-free patients are determined. Therapy is deemed to be effective if there is a statistically significant difference in the rate or proportion of exacerbation-free patients between the treated group and the placebo group for either of these measurements. In addition, time to first exacerbation and exacerbation duration and severity may also be measured. A measure of effectiveness as therapy in this regard is a statistically significant difference in the time to first exacerbation or duration and severity in the treated group compared to control group.

MRI can be used to measure active lesions using gadolinium-DTPA-enhanced imaging (McDonald et al. Ann. Neurol. 36:14, 1994) or the location and extent of lesions using T₂ -weighted techniques. Briefly, baseline MRIs are obtained. The same imaging plane and patient position are used for each subsequent study. Positioning and imaging sequences can be chosen to maximize lesion detection and facilitate lesion tracing. The same positioning and imaging sequences can be used on subsequent studies. The presence, location and extent of MS lesions can be determined by radiologists. Areas of lesions can be outlined and summed slice by slice for total lesion area. Three analyses may be done: evidence of new lesions, rate of appearance of active lesions, percentage change in lesion area (Paty et al., Neurology 43:665, 1993). Improvement due to therapy can be established by a statistically significant improvement in an individual patient compared to baseline or in a treated group versus a placebo group.

Exemplary symptoms associated with multiple sclerosis include: optic neuritis, diplopia, nystagmus, ocular dysmetria, internuclear ophthalmoplegia, movement and sound phosphenes, afferent pupillary defect, paresis, monoparesis, paraparesis, hemiparesis, quadraparesis, plegia, paraplegia, hemiplegia, tetraplegia, quadraplegia, spasticity, dysarthria, muscle atrophy, spasms, cramps, hypotonia, clonus, myoclonus, myokymia, restless leg syndrome, footdrop, dysfunctional reflexes, paraesthesia, anaesthesia, neuralgia, neuropathic and neurogenic pain, l'hermitte's, proprioceptive dysfunction, trigeminal neuralgia, ataxia, intention tremor, dysmetria, vestibular ataxia, vertigo, speech ataxia, dystonia, dysdiadochokinesia, frequent micturation, bladder spasticity, flaccid bladder, detrusor-sphincter dyssynergia, erectile dysfunction, anorgasmy, frigidity, constipation, fecal urgency, fecal incontinence, depression, cognitive dysfunction, dementia, mood swings, emotional lability, euphoria, bipolar syndrome, anxiety, aphasia, dysphasia, fatigue, uhthoffs symptom, gastroesophageal reflux, and sleeping disorders.

Candidate patients for prevention may be identified by the presence of genetic factors. For example, a majority of MS patients have HLA-type DR2a and DR2b. The MS patients having genetic dispositions to MS who are suitable for treatment fall within two groups. First are patients with early disease of the relapsing remitting type. Entry criteria can include disease duration of more than one year, EDSS score of 1.0 to 3.5, exacerbation rate of more than 0.5 per year, and free of clinical exacerbations for 2 months prior to study. The second group would include people with disease progression greater than 1.0 EDSS unit/year over the past two years. Candidate patients for prevention may be identified by evaluating cytokine parameters, e.g., an IL-10 or IL-21 parameter.

Efficacy of the IL-21/IL-21R agonist in the context of prevention is judged based on the following criteria: frequency of MBP reactive T cells determined by limiting dilution, proliferation response of MBP reactive T cell lines and clones, cytokine profiles of T cell lines and clones to MBP established from patients. Efficacy is established by decrease in frequency of reactive cells, a reduction in thymidine incorporation with altered peptide compared to native, and a reduction in TNF and IFN-α. Clinical measurements include the relapse rate in one and two-year intervals, and a change in EDSS, including time to progression from baseline of 1.0 unit on the EDSS which persists for six months. On a Kaplan-Meier curve, a delay in sustained progression of disability shows efficacy. Other criteria include a change in area and volume of T2 images on MRI, and the number and volume of lesions determined by gadolinium enhanced images.

In one embodiment, the IL-21/IL-21R agonists, e.g., pharmaceutical compositions thereof, are administered in combination therapy, i.e., combined with other agents, e.g., therapeutic agents, which are useful for treating pathological conditions or disorders, such as immune and inflammatory disorders of the brain, e.g., multiple sclerosis. The term “in combination” in this context means that the agents are given substantially contemporaneously, either simultaneously or sequentially. If given sequentially, at the onset of administration of the second compound, the first of the two compounds is preferably still detectable at effective concentrations at the site of treatment.

For example, the combination therapy can include one or more IL-21/IL-21R agonists (e.g., an IL-21 polypeptide or fusion protein, a peptide agonist or a small molecule agonist) co-formulated with, and/or co-administered with, one or more additional therapeutic agents, e.g., one or more cytokine and growth factor inhibitors, immunosuppressants, anti-inflammatory agents, metabolic inhibitors, enzyme inhibitors, and/or cytotoxic or cytostatic agents, as described in more detail below. Furthermore, one or more IL-21/IL-21R agonists described herein may be used in combination with two or more of the therapeutic agents described herein. Such combination therapies may advantageously utilize lower dosages of the administered therapeutic agents, thus avoiding possible toxicities or complications associated with the various monotherapies. Moreover, the therapeutic agents disclosed herein act on pathways that differ from the IL-21/IL-21 R receptor pathway, and thus are expected to enhance and/or synergize with the effects of the IL-21/IL-21R agonists. Preferred therapeutic agents used in combination with an IL-21/IL-21R agonist are those agents that interfere at different stages in the autoimmune and subsequent inflammatory response.

Non-limiting examples of agents for treating or preventing multiple sclerosis with which an IL-21-/IL21R agonist can be combined include the following: interferons, e.g., interferon-beta-1α (e.g., AVONEX™; Biogen) and interferon-1β (BETASERON™; human interferon β substituted at position 17; Berlex/Chiron); glatiramer acetate (also termed Copolymer 1, Cop-1; COPAXONE™; Teva Pharmaceutical Industries, Inc.); hyperbaric oxygen; intravenous immunoglobulin; clabribine; TNF antagonists as described herein; corticosteroids; prednisolone; methylprednisolone; azathioprine; cyclophosphamide; cyclosporine; methotrexate; 4-aminopyridine; and tizanidine. Additional antagonists that can be used in combination with IL-21 agonists include antibodies to, or antagonists of, other human cytokines or growth factors, for example, TNF, LT, IL-1, IL-2, IL-6, IL-7, IL-8, IL-12 IL-15, IL-16, IL-18, EMAP-11, GM-CSF, FGF, and PDGF. IL-21 agonists as described herein can be combined with antibodies to cell surface molecules such as CD2, CD3, CD4, CD8, CD25, CD28, CD30, CD40, CD45, CD69, CD80, CD86, CD90 or their ligands. The IL-21 agonists may also be combined with agents, such as methotrexate, cyclosporine, FK506, rapamycin, mycophenolate mofetil, leflunomide, NSAIDs, for example, ibuprofen, corticosteroids such as prednisolone, phosphodiesterase inhibitors, adenosine agonists, antithrombotic agents, complement inhibitors, adrenergic agents, agents which interfere with signaling by proinflammatory cytokines as described herein, IL-1β converting enzyme inhibitors (e.g., Vx740), anti-P7s, PSGL, TACE inhibitors, T-cell signaling inhibitors such as kinase inhibitors, metal loproteinase inhibitors, sulfasalazine, azathloprine, 6-mercaptopurines, angiotensin converting enzyme inhibitors, soluble cytokine receptors and derivatives thereof, as described herein, and anti-inflammatory cytokines (e.g. IL-4, IL-10, IL-13 and TGF).

Examples of therapeutic agents for treating or preventing multiple sclerosis with which the IL-21/IL-21R agonists can be combined include interferon-β, for example, IFN β-1α and IFN β-1β; glatiramer acetate (e.g, COPAXONE™), corticosteroids, IL-1 inhibitors, TNF inhibitors, antibodies to CD40 ligand and CD80, and IL-12 antagonists. Additional examples include agents that may be used to treat one or more symptoms or side effects of MS, e.g., amantadine, baclofen, mineral oil, papaverine, meclizine, hydroxyzine, sulfamethoxazole, ciprofloxacin, docusate, ciprofloxacin, pemoline, dantrolene, desmopressin, desmopressin, dexamethasone, prednisone, tolterodine, phenytoin, oxybutynin, oxybutynin (extended release formula), bisacodyl, venlafaxine, amitriptyline, docusate stool softener laxative, sodium phosphate, methenamine, baclofen (intrathecal), clonazepam, isoniazid, vardenafil, nitrofurantoin, psyllium hydrophilic mucilloid, alprostadil, gabapentin, mitoxantrone, oxybutynin, nortriptyline, paroxetine, magnesium hydroxide, propantheline bromide, alprostadil, modafinil, fluoxetine, phenazopyridine, glycerin, methylprednisolone, carbamazepine, imipramine, diazepam, sildenafil, bupropion, tizanidine, and sertraline.

Examples of those agents include IL-12 antagonists, such as chimeric, humanized, human or in vitro generated antibodies (or antigen-binding fragments thereof) that bind to IL-12 (preferably human IL-12), e.g., the antibody disclosed in WO 00/56772, Genetics Institute/BASF); IL-12 receptor inhibitors, e.g., antibodies to human IL-12 receptor; and soluble fragments of the IL-12 receptor, e.g., human IL-12 receptor. Examples of IL-15 antagonists include antibodies (or antigen-binding fragments thereof) against IL-15 or its receptor, e.g., chimeric, humanized, human or in vitro generated antibodies to human IL-15 or its receptor, soluble fragments of the IL-15 receptor, and IL-15-binding proteins. Examples of IL-18 antagonists include antibodies, e.g., chimeric, humanized, human or in vitro generated antibodies (or antigen-binding fragments thereof), to human IL-18, soluble fragments of the IL-18 receptor, and IL-18 binding proteins (IL-18BP, Mallet et al. (2001) Circ. Res. 28). Examples of IL-1 antagonists include Interleukin-1-converting enzyme (ICE) inhibitors, such as Vx740, IL-1 antagonists, e.g., IL-1RA (ANIKINRA, AMGEN), sIL1RII (Immunex), and anti-IL-1 receptor antibodies (or antigen-binding fragments thereof).

Examples of TNF antagonists include chimeric, humanized, human or in vitro generated antibodies (or antigen-binding fragments thereof) to TNF (e.g., human TNF a), such as D2E7, (human TNFa antibody, U.S. Pat. No. 6,258,562; BASF), CDP-571/CDP-870/BAY-10-3356 (humanized anti-TNFa antibody; Celltech/Pharmacia), cA2 (chimeric anti-TNFa antibody; Remicade™, Centocor); anti-TNF antibody fragments (e.g., CPD870); soluble fragments of the TNF receptors, e.g., p55 or p75 human TNF receptors or derivatives thereof, e.g., 75 kdTNFR-IgG (75 kD TNF receptor-IgG fusion protein, ENBREL™; Immunex; see e.g., Arthritis & Rheumatism (1994) Vol. 37, S295; J. Invest. Med. (1996) Vol. 44, 235A), p55 kdTNFR-IgG (55 kD TNF receptor-IgG fusion protein (LENERCEPT™)); enzyme antagonists, e.g., TNFa converting enzyme (TACE) inhibitors (e.g., an alpha-sulfonyl hydroxamic acid derivative, WO 01/55112, and N-hydroxyformamide TACE inhibitor GW 3333, -005, or -022); and TNF-bp/s-TNFR (soluble TNF binding protein; see e.g., Arthritis & Rheumatism (1996) Vol. 39, No. 9 (supplement), S284; Amer. J. Physiol.—Heart and Circulatory Physiology (1995) Vol. 268, pp. 37-42). Preferred TNF antagonists are soluble fragments of the TNF receptors, e.g., p55 or p75 human TNF receptors or derivatives thereof, e.g., 75 kdTNFR-IgG, and TNFa converting enzyme (TACE) inhibitors.

In other embodiments, the IL-21-/IL21R agonists described herein can be administered in combination with one or more of the following: IL-13 antagonists, e.g., soluble IL-13 receptors (sIL-13) and/or antibodies against IL-13; IL-2 antagonists, e.g., DAB 486-IL-2 and/or DAB 389-IL-2 (IL-2 fusion proteins; Seragen; see e.g., Arthritis & Rheumatism (1993) Vol. 36, 1223), and/or antibodies to IL-2R, e.g., anti-Tac (humanized anti-IL-2R; Protein Design Labs, Cancer Res. 1990 Mar 1; 50(5):1495-502). Yet another combination includes IL-21 antagonists in combination with non-depleting anti-CD4 inhibitors (IDEC-CE9.1/SB 210396 (non-depleting primatized anti-CD4 antibody; IDEC/SmithKline). Yet other preferred combinations include antagonists of the co-stimulatory pathway CD80 (B7.1) or CD86 (B7.2) including antibodies, soluble receptors or antagonistic ligands; as well as p-selectin glycoprotein ligand (PSGL), anti-inflammatory cytokines, e.g., IL-4 (DNAX/Schering); IL-10 (SCH 52000; recombinant IL-10 DNAX/Schering); IL-13 and TGF, and agonists thereof (e.g., agonist antibodies).

In other embodiments, one or more IL-21-/IL21R agonists can be co-formulated with, and/or co-administered with, one or more anti-inflammatory drugs, immunosuppressants, or metabolic or enzymatic inhibitors. Non-limiting examples of the drugs or inhibitors that can be used in combination with the IL-21 agonists described herein, include, but are not limited to, one or more of: non-steroidal anti-inflammatory drug(s) (NSAIDs), e.g., ibuprofen, Tenidap (see e.g., Arthritis & Rheumatism (1996) Vol. 39, No. 9 (supplement), S280)), Naproxen (see e.g., Neuro Report (1996) Vol. 7, pp. 1209-1213), Meloxicam, Piroxicam, Diclofenac, and Indomethacin; Sulfasalazine (see e.g., Arthritis & Rheumatism (1996) Vol. 39, No. 9 (supplement), S281); corticosteroids such as prednisolone; cytokine suppressive anti-inflammatory drug(s) (CSAIDs); inhibitors of nucleotide biosynthesis, e.g., inhibitors of purine biosynthesis, folate antagonists (e.g., methotrexate (N-[4-[[(2,4-diamino-6-pteridinyl)methyl]methylamino]benzoyl]-L-glutamic acid); and inhibitors of pyrimidine biosynthesis, e.g., dihydroorotate dehydrogenase (DHODH) inhibitors (e.g., leflunomide (see e.g., Arthritis & Rheumatism (1996) Vol. 39, No. 9 (supplement), S131; Inflammation Research (1996) Vol. 45, pp. 103-107). Preferred therapeutic agents for use in combination with IL-21/IL-21R antagonists include NSAIDs, CSAIDs, (DHODH) inhibitors (e.g., leflunomide), and folate antagonists (e.g., methotrexate).

Examples of additional inhibitors include one or more of: corticosteroids (oral, inhaled and local injection); immunosuppresants, e.g., cyclosporin, tacrolimus (FK-506); and mTOR inhibitors, e.g., sirolimus (rapamycin) or rapamycin derivatives, e.g., soluble rapamycin derivatives (e.g., ester rapamycin derivatives, e.g., CCI-779 (Elit. L. (2002) Current Opinion Investig. Drugs 3(8):1249-53; Huang, S. et al. (2002) Current Opinion Investig. Drugs 3(2):295-304); agents which interfere with signaling by proinflammatory cytokines such as TNFa or IL-1 (e.g. IRAK, NIK, IKK, p38 or MAP kinase inhibitors); COX2 inhibitors, e.g., celecoxib and variants thereof, MK-966, see e.g., Arthritis & Rheumatism (1996) Vol. 39, No. 9 (supplement), S81); phosphodiesterase inhibitors, e.g., R973401 (phosphodiesterase Type IV inhibitor; see e.g., Arthritis & Rheumatism (1996) Vol. 39, No. 9 (supplement), S282)); phospholipase inhibitors, e.g., inhibitors of cytosolic phospholipase 2 (cPLA2) (e.g., trifluoromethyl ketone analogs (U.S. Pat. No. 6,350,892)); inhibitors of vascular endothelial cell growth factor or growth factor receptor, e.g., VEGF inhibitor and/or VEGF-R inhibitor; and inhibitors of angiogenesis. Preferred therapeutic agents for use in combination with IL-21/IL-21R antagonists immunosuppresants, e.g., cyclosporin, tacrolimus (FK-506); and mTOR inhibitors, e.g., sirolimus (rapamycin) or rapamycin derivatives, e.g., soluble rapamycin derivatives (e.g., ester rapamycin derivatives, e.g., CCI-779; COX2 inhibitors, e.g., celecoxib and variants thereof; and phospholipase inhibitors, e.g., inhibitors of cytosolic phospholipase 2 (cPLA2) (e.g., trifluoromethyl ketone analogs)

Additional examples of therapeutic agents that can be combined with an IL-21/IL-21R agonist include one or more of: 6-mercaptopurines (6-MP); azathioprine sulphasalazine; mesalazine; olsalazine chloroquinine/hydroxychloroquine; pencillamine; aurothiornalate (intramuscular and oral); azathioprine; cochicine; beta-2 adrenoreceptor agonists (salbutamol, terbutaline, salmeteral); xanthines (theophylline, aminophylline); cromoglycate; nedocromil; ketotifen; ipratropium and oxitropium; mycophenolate mofetil; adenosine agonists; antithrombotic agents; complement inhibitors; and adrenergic agents.

Another aspect of the present invention accordingly relates to kits for carrying out the combined administration of the IL-21/IL21R antagonists with other therapeutic compounds. In one embodiment, the kit comprises one or more binding agents formulated in a pharmaceutical carrier, and at least one agent, e.g., therapeutic agent, formulated as appropriate, in one or more separate pharmaceutical preparations.

Assays for Evaluating Cytokine Levels

Any standard assay can be used to evaluate cytokine levels in a sample or a subject. For example, the sample can be obtained from a subject or can include culture cells. Exemplary samples can be obtained or derived from one or more cells, tissue, or bodily fluids such as blood, urine, lymphatic fluid, cerebrospinal fluid, or amniotic fluid, cultured cells (e.g., tissue culture cells), buccal swabs, mouthwash, stool, tissues slices, and biopsy materials (e.g., biopsy aspiration).

Methods for evaluating cytokine levels include evaluating nucleic acids to detect mRNA or cDNA encoding a cytokine of interest (e.g., IL-10 or IL-21) or evaluating proteins to detect the cytokine itself. Nucleic acids can be evaluated, e.g., using RT-PCR (e.g., quantitative PCR) or nucleic acid microarrays. Proteins can be evaluated, e.g., using mass spectroscopy or an immunoassay.

ELISAs provide one convenient form of immunoassay. For example, Biosource International, Camarillo Calif. provides assay reagents that can be used to detect IL-10 with a sensitivity of <0.2 pg/ml and to IL-12 with a sensitivity of <2 pg/ml). Similarly, R&D Systems provides reagents to detect IFN-γ with a sensitivity <8 pg/ml or TGF-beta1 with a sensitivity of <7 pg/ml.

SEARCHLIGHT™ Proteome Array System (Pierce, Boston Technology Center) provides comprehensive reagents for evaluating multiple cytokines at once.

These methods can be used to evaluate administration of an IL-21/IL-21R agonist. For example, to determine if such agonist causes a statistically significant change in the levels of a cytokine, e.g., IL-10 or IFNγ or to determine if it causes an acceptable changes, e.g., to a level in a range of normal of a cytokine, e.g., IL-10 or IFNγ. Information from the evaluating can be used to modulate the dosage of the agonist. For example, if IL-10 levels are not increased to levels within the range of a normal subject, administration of the agonist can be increased, e.g., by increasing dosage or frequency, e.g., by a proportional or corresponding amount, or by at least about 1.5, 1.8, or 2 fold. Conversely, if IL-10 levels are increased beyond the desired range, the administration of the agonist can be decreased, e.g., by decreasing dosage or frequency, e.g., by a proportional or corresponding amount, or by at least 20, 30, 40, 50, or 75%.

Assays for Evaluating the Activity of IL-21/IL21R Agonists as Immune Activators

The activity of IL-21/IL21R agonists as activators of an immune system can, among other means, be measured by the following methods: Suitable assays for thymocyte or splenocyte cytotoxicity include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function 3.1-3.19; Chapter 7, Immunologic studies in Humans); Herrmann et al., Proc. Natl. Acad. Sci. U.S.A. 78:2488-2492, 1981; Herrmann et al., J. Immunol. 128:1968-1974, 1982; Handa et al., J. Immunol. 135:1564-1572, 1985; Takai et al., J. Immunol. 137:3494-3500, 1986; Takai et al., J. Immunol. 140:508-512, 1988; Herrmann et al., Proc. Natl. Acad. Sci. U.S.A. 78:2488-2492, 1981; Herrmann et al., J. Immunol. 128:1968-1974, 1982; Handa et al., J. Immunol. 135:1564-1572, 1985; Takai et al., J. Immunol. 137:3494-3500, 1986; Bowmanet al., J. Virology 61:1992-1998; Takai et al., J. Immunol. 140:508-512, 1988; Bertagnolli et al., Cellular Immunology 133:327-341, 1991; Brown et al., J. Immunol. 153:3079-3092, 1994.

Assays for T-cell-dependent immunoglobulin responses and isotype switching (which will identify, among others, proteins that modulate T-cell dependent antibody responses and that affect Th1/Th2 profiles) include, without limitation, those described in: Maliszewski, J. Immunol. 144:3028-3033, 1990; and Assays for B cell function: In vitro antibody production, Mond, J. J. and Brunswick, M. In Current Protocols in Immunology. J. E. e.a. Coligan eds. Vol 1 pp. 3.8.1-3.8.16, John Wiley and Sons, Toronto. 1994.

Mixed lymphocyte reaction (MLR) assays (which will identify, among others, proteins that generate predominantly Th1 and CTL responses) include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function 3.1-3.19; Chapter 7, Immunologic studies in Humans); Takai et al., J. Immunol. 137:3494-3500, 1986; Takai et al., J. Immunol. 140:508-512, 1988; Bertagnolli et al., J. Immunol. 149:3778-3783, 1992.

Dendritic cell-dependent assays (which will identify, among others, proteins expressed by dendritic cells that activate naive T-cells) include, without limitation, those described in: Guery et al., J. Immunol. 134:536-544, 1995; Inaba et al., Journal of Experimental Medicine 173:549-559, 1991; Macatonia et al., Journal of Immunology 154:5071-5079, 1995; Porgador et al., Journal of Experimental Medicine 182:255-260, 1995; Nair et al., Journal of Virology 67:4062-4069, 1993; Huang et al., Science 264:961-965, 1994; Macatonia et al., Journal of Experimental Medicine 169:1255-1264, 1989; Bhardwaj et al., Journal of Clinical Investigation 94:797-807, 1994; and Inaba et al., Journal of Experimental Medicine 172:631-640, 1990.

Assays for lymphocyte survival/apoptosis (which will identify, among others, proteins that prevent apoptosis after superantigen induction and proteins that regulate lymphocyte homeostasis) include, without limitation, those described in: Darzynkiewicz et al., Cytometry 13:795-808, 1992; Gorczyca et al., Leukemia 7:659-670, 1993; Gorczyca et al., Cancer Research 53:1945-1951, 1993; Itoh et al., Cell 66:233-243, 1991; Zacharchuk, Journal of Immunology 145:4037-4045, 1990; Zamai et al., Cytometry 14:891-897, 1993; Gorczyca et al., International Journal of Oncology 1:639-648, 1992.

Assays for proteins that influence early steps of T-cell commitment and development include, without limitation, those described in: Antica et al., Blood 84:111-117, 1994; Fine et al., Cellular Immunology 155:111-122, 1994; Galy et al., Blood 85:2770-2778, 1995; Toki et al., Proc. Nat. Acad Sci. U.S.A. 88:7548-7551, 1991.

Assays for evaluating activation of STAT are described, e.g., in Gilmour et al. (1996) Proc. Natl. Acad. Sci. USA 92:10772-10776. For example, evaluated cells (e.g., cells treated with an agonist or a candidate agonist) can be lysed and tyrosine phosphorylated proteins can be immunoprecipitated with an anti-phosphotyrosine antibody. Then precipitated materials can then be evaluated using antibodies specific for a signalling pathway component, e.g., an antibody to the STAT protein, e.g., STAT5.

Assays for Measuring the Activity of IL-21/IL21R Agonists as Modulators of Cytokine Production and Cell Proliferation/Differentiation

The activity of IL-21/IL21R agonists as modulator of cytokine production and cell proliferation/differentiation can be tested using any one of a number of routine factor dependent cell proliferation assays for cell lines including, without limitation, 32D, DA2, DA1G, T10, B9, B9/11, BaF3, MC9/G, M+(preB M+), 2E8, RB5, DA1, 123, T1165, HT2, CTLL2, TF-1, Mo7e and CMK.

Assays for T-cell or thymocyte proliferation include without limitation those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function 3.1-3.19; Chapter 7, Immunologic studies in Humans); Takai et al., J. Immunol. 137:3494-3500, 1986; Bertagnolli et al., J. Immunol. 145:1706-1712, 1990; Bertagnolli et al., Cellular Immunology 133:327-341, 1991; Bertagnolli, et al., J. Immunol. 149:3778-3783, 1992; Bowman et al., J. Immunol. 152: 1756-1761, 1994.

Assays for cytokine production and/or proliferation of spleen cells, lymph node cells or thymocytes include, without limitation, those described in: Polyclonal T cell stimulation, Kruisbeek, A. M. and Shevach, E. M. In Current Protocols in Immunology. J. E. e.a. Coligan eds. Vol 1 pp. 3.12.1-3.12.14, John Wiley and Sons, Toronto. 1994; and Measurement of mouse and human Interferon gamma, Schreiber, R. D. In Current Protocols in Immunology. J. Coligan eds. Vol 1 pp. 6.8.1-6.8.8, John Wiley and Sons, Toronto. 1994.

Assays for proliferation and differentiation of hematopoietic and lymphopoietic cells include, without limitation, those described in: Measurement of Human and Murine Interleukin 2 and Interleukin 4, Bottomly, K., Davis, L. S. and Lipsky, P. E. In Current Protocols in Immunology. J. Coligan eds. Vol 1 pp. 6.3.1-6.3.12, John Wiley and Sons, Toronto. 1991; deVries et al., J. Exp. Med. 173:1205-1211, 1991; Moreau et al., Nature 336:690-692, 1988; Greenberger et al., Proc. Natl. Acad. Sci. U.S.A. 80:2931-2938, 1983; Measurement of mouse and human interleukin 6-Nordan, R. In Current Protocols in Immunology. J. E. e.a. Coligan eds. Vol 1 pp. 6.6.1-6.6.5, John Wiley and Sons, Toronto. 1991; Smith et al., Proc. Natl. Acad. Sci. U.S.A. 83:1857-1861, 1986; Measurement of human Interleukin 11-Bennett, F., Giannotti, J., Clark, S. C. and Turner, K. J. In Current Protocols in Immunology. J. E. e.a. Coligan eds. Vol 1 pp. 6.15.1 John Wiley and Sons, Toronto. 1991; Measurement of mouse and human Interleukin 9-Ciarletta, A., Giannotti, J., Clark, S. C. and Turner, K. J. In Current Protocols in Immunology. J. Coligan eds. Vol 1 pp. 6.13.1, John Wiley and Sons, Toronto. 1991.

Assays for T-cell clone responses to antigens (which will identify, among others, proteins that affect APC-T cell interactions as well as direct T-cell effects by measuring proliferation and cytokine production) include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function; Chapter 6, Cytokines and their cellular receptors; Chapter 7, Immunologic studies in Humans); Weinberger et al., Proc. Natl. Acad. Sci. U.S.A. 77:6091-6095, 1980; Weinberger et al., Eur. J. Immun. 11:405-411, 1981; Takai et al., J. Immunol. 137:3494-3500, 1986; Takai et al., J. Immunol. 140:508-512, 1988.

In the following non-limiting example, we demonstrate, inter alia, that IL-21 results in partial protection in EAE mice.

### EXAMPLE

### IL-21 Results in Partial Protection in EAE Mice

Reagents

Mice

Female C57BL/6 and female SJL/J mice were obtained from The Jackson Laboratory. The animals were housed within an AAALAC-approved barrier facility and monitored for parasites as well as bacterial and viral pathogens. All mice were used at 8-10 weeks of age.

Proliferative Response

Lymph node cells of C57BL/6 female mice challenged with the mouse oligodendrocyte glycoprotein (MOG)₃₅₋₅₅ peptide were harvested from the mice 20 days after immunization. Cells were cultured in Dulbecco's Modified Eagle's Media (DME) containing 10% fetal calf serum (FCS). The cells were restimulated with the MOG₃₅₋₅₅ peptide (25 μg/ml) and grown in flat-bottom 96-well microtiter plates in the presence or absence of murine IL-21. After 72 hours, the plates were pulsed with 0.5 μCi tritiated thymidine/well and incubated for a 4- to 6-hour period. The mean incorporation of thymidine in the DNA in the triplicate wells was measured by a scintillation counter.

Cytokine Quantitation

Lymph node cells derived from immunized mice were activated in vitro with 25 μg/ml of the MOG₃₅₋₅₅ peptide. Cells were cultured in (DME) containing 10% FCS and IL-2 (10 U/ml) and various concentrations of murine IL-21 ranging from 5 ng/ml to 25 ng/ml. Culture supernatants were collected after 72 hours and were analyzed for cytokine and chemokine production using the SEARCHLIGHT™ Proteome Array System (Pierce, Boston Technology Center).

Induction and Assessment of Experimental Autoimmune Encephalymyelitis (EAE)

Female SJL/J mice were immunized subcutaneously with 100 μg of the proteolipid protein (PLP)₁₃₉-151 peptide emulsified in complete Freund's adjuvant supplemented with 800 μg of Mycobacterium tuberculosis H37 Ra (Difco Laboratories). Mice also received intraperitoneal injections with 400 ng of pertussis toxin (List Biological Laboratories) at the time of immunization and 48 hours later. Animals were scored daily for clinical signs of EAE according to the following scale: 0, no disease, 1, limp tail; 2, hind limb weakness or partial paralysis; 3, complete hind limb paralysis; 4, front and hind limb paralysis, and 5, moribund.

The mean clinical score was calculated by averaging the individual scores of the mice in each group.

IL-21 Administration in Vivo

Mice sensitized for EAE were given intraperitoneal injections of murine IL-21 at 100 ng/day or 1 μg/day in 0.2 ml of PBS. The control mice were dosed with 0.2 ml of saline. Treatment began on the day prior to immunization with the PLP₁₃₉-151 peptide and continued on alternate days for a total of ten doses.

Proliferative Response and Cytokine Production Induced by Murine IL-21

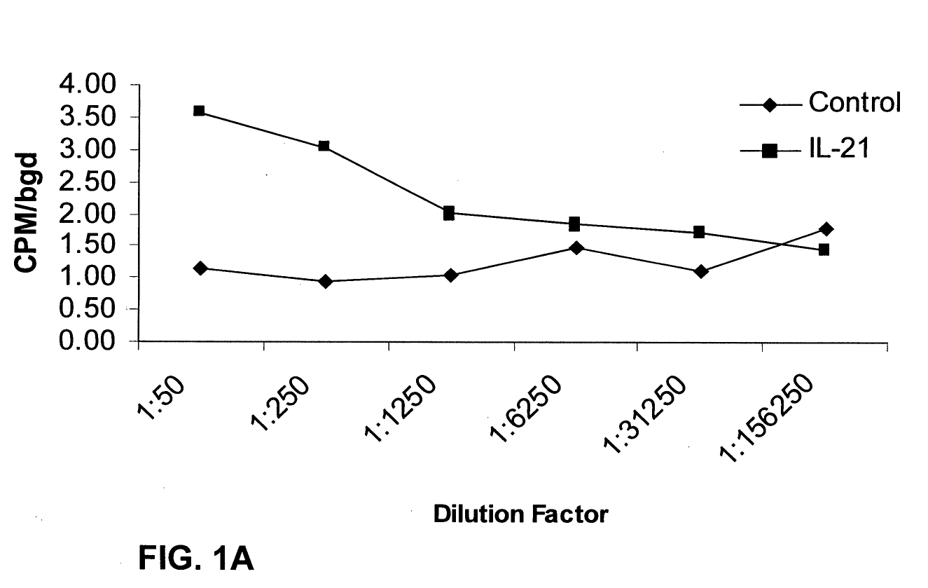

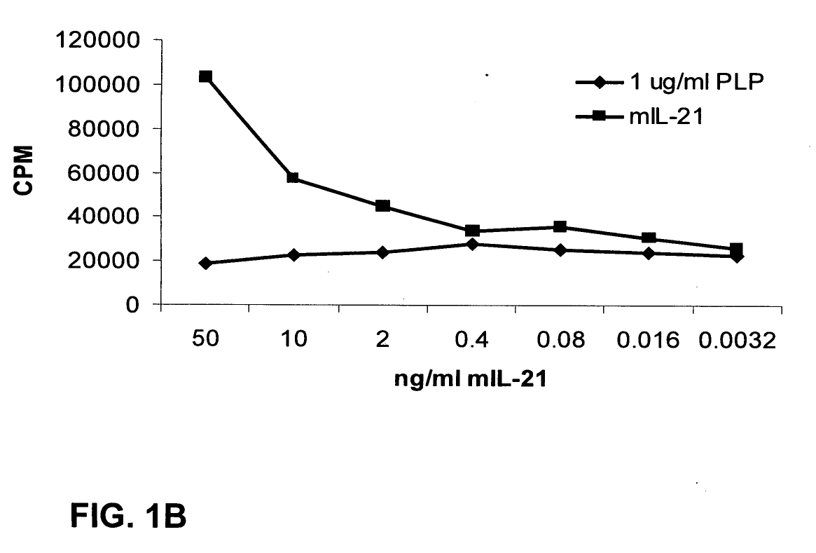

The effect of IL-21 on the induction and expansion of MOG₃₅₋₅₅-specific T cell responses was evaluated. The results indicate that IL-21 induces proliferation of lymphocytes in a dose dependent manner. In FIG. 1A and Table 1, lymphocytes cultured with 20 ng/ml of IL-21 showed a significant 3.5-fold increase in proliferation as compared to cells stimulated with peptide alone. Upon titration of the cytokine, the cells exhibited a similar proliferative capacity as the control cells.

TABLE 1
Lymphocyte proliferation in the presence of IL-21

| | Control | IL-21 | |
|---|---|---|---|
| Dilution | CPM/bgd | CPM/bgd | ng/ml |
| 1:50 | 1.14 | 3.57 | 20 |
| 1:250 | 0.95 | 3.01 | 4 |
| 1:1250 | 1.04 | 2.02 | 0.8 |
| 1:6250 | 1.49 | 1.86 | 0.16 |
| 1:31250 | 1.13 | 1.70 | 0.032 |
| 1:156250 dil | 1.77 | 1.45 | 0.0064 |

FIG. 1B shows increased proliferation of T cells from proteolipid protein (PLP) transgenic mice cultured at the indicated concentration of murine IL-21 (ng/ml) compared to cells treated with only 1 μg/ml of PLP.

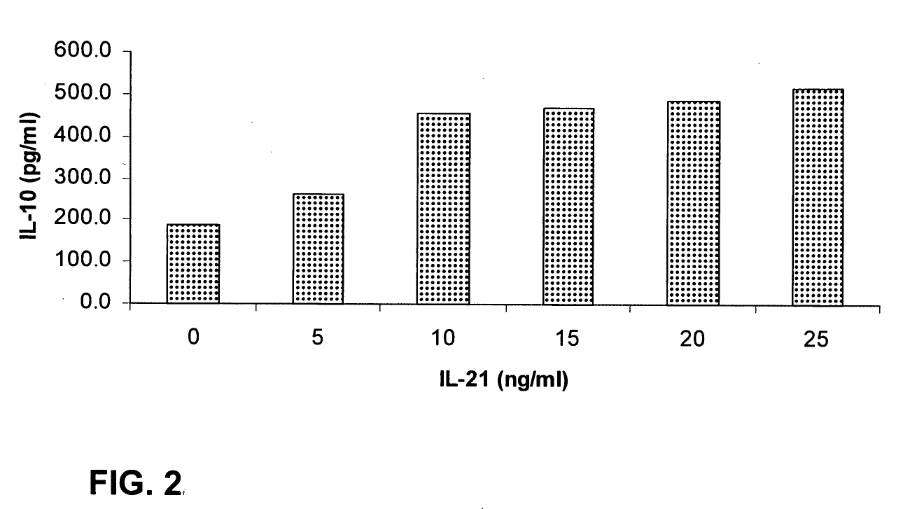

To identify whether the proliferative T cell response could be correlated with cytokine production, lymphocytes were cultured with IL-21. IL-21 induced increased secretion of the Th2 cytokine, IL-10, as compared to untreated cells. This response was saturable (FIG. 2 and Table 2) and at the highest tested concentration of IL-21 (25 ng/ml), cells produced approximately 2.5-fold more IL-10 than the control group.

TABLE 2
IL-21 induces IL-10 secretion

| IL-21 | IL-10 detected |
|---|---|
| (ng/ml) | (pg/ml) |
| 0 | 189.3 |
| 5 | 260.8 |
| 10 | 457.3 |
| 15 | 466.8 |
| 20 | 486.1 |
| 25 | 515.0 |

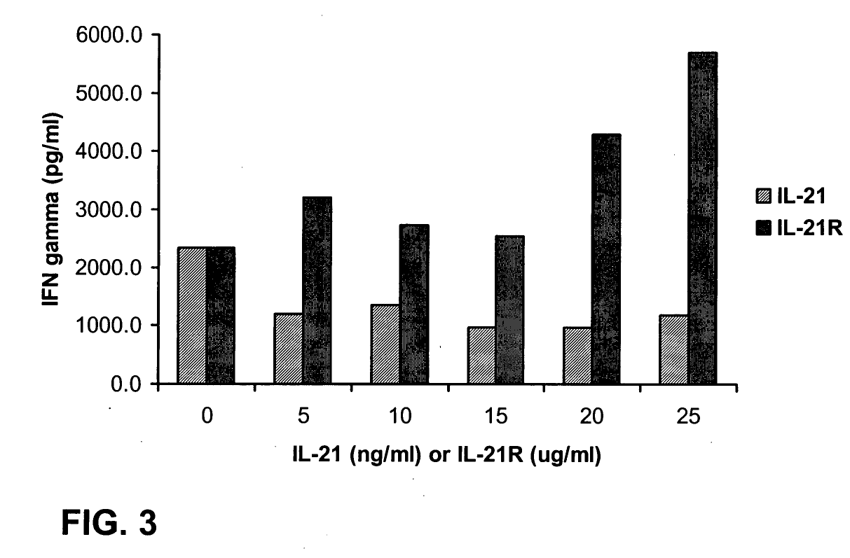

FIG. 3 shows a decrease in secretion of IFNγ by spleen cells treated with the indicated concentrations of murine IL-21 compared to control cells. Addition of IL-21 to MOG 33-35-stimulated spleen cells from immunized mice results in a two-fold decrease of IFNγ, whereas addition of IL-21R yields a two-fold increase. When lymph node cells were treated with IL-21, IFNγ levels were decreased from 20,000 pg/ml (control) or 15840 pg/ml (mock treated) to 3260 pg/ml (IL-21 treated).

The changes in cytokine secretion upon IL-21 or IL-21R treatment are summarized as follows in Table 3.

TABLE 3
Changes in Cytokine Secretion

| | IL-21 Treatment | IL-21R Treatment |
|---|---|---|
| Increase | IL-10 | IL-12, IFNγ, TGFβ |
| Decrease | IL-1α, IL-2, IL-6, | None |
| | IFNγ, IL-18 | |
| No Change | IL-1β, IL-4, IL-5, | IL-1α, IL-1β, IL-2, |
| | IL-12, IL-13, TNFα, | IL-4, IL-5, IL-6, IL-10, IL-13, |
| | TGFβ, MIP-1α, GM-CSF | IL-18, TNFα, MIP-1α, |
| | | GM-CSF |

Development of EAE in Mice Treated with IL-21

To determine the role of IL-21 in the development of EAE, mice were immunized with the encephalitogenic PLP₁₃₉-151 peptide in CFA plus pertussis toxin and treated them with a prophylactic regimen of IL-21. The clinical course of EAE was compared in mice treated with saline or IL-21.

Table 4 documents average changes in cytokine secretion in two exemplary PLP cultures treated in the presence or absence of murine IL-21. The cytokine level in the untreated control cells is normalized to 100.

TABLE 4

| | | Treated w/ |
|---|---|---|
| Cytokine | Control | mIL-21 |
| IL2 | 100 | 59 |
| IL5 | 100 | 73 |
| IL6 | 100 | 56 |
| IL10 | 100 | 250 |
| IFNg | 100 | 39 |
| TNFa | 100 | 56 |
| GMCSF | 100 | 15 |

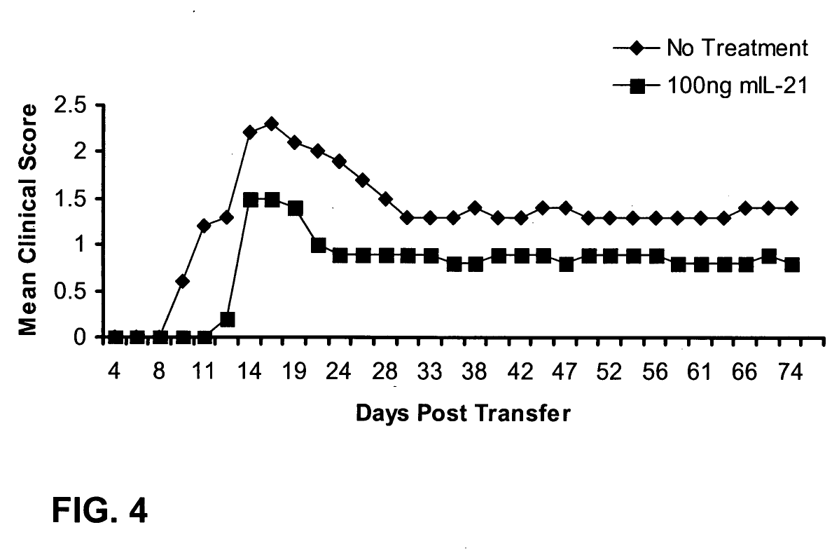

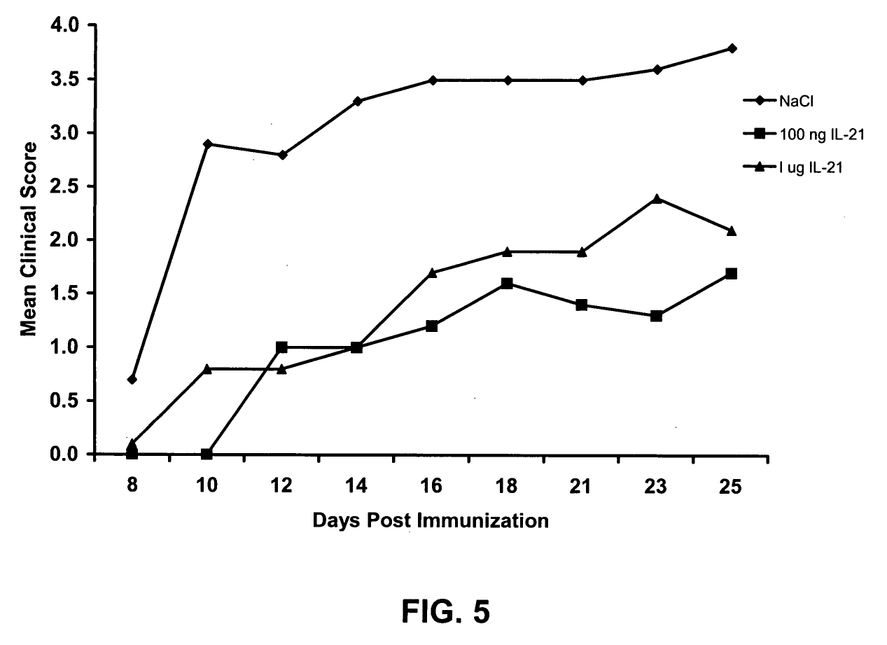

As shown in FIGS. 4 and 5, control mice were highly susceptible to disease. In contrast, mice treated with either a low (100 ng/day) or high (1 μg/day) dose of IL-21 had less severe clinical scores.

FIG. 5 and Table 5 shows a decrease in the severity of EAE in mice treated with either a low (100 ng/day) or high (1 μg/day) dose of murine IL-21 compared to control mice.

TABLE 5

| Day | NaCl | 100 ng IL-21 | 1 μg IL-21 |
|---|---|---|---|
| 8 | 0.7 | 0.0 | 0.1 |
| 10 | 2.9 | 0.0 | 0.8 |
| 12 | 2.8 | 1.0 | 0.8 |
| 14 | 3.3 | 1.0 | 1.0 |
| 16 | 3.5 | 1.2 | 1.7 |
| 18 | 3.5 | 1.6 | 1.9 |
| 21 | 3.5 | 1.4 | 1.9 |
| 23 | 3.6 | 1.3 | 2.4 |
| 25 | 3.8 | 1.7 | 2.1 |

Our findings indicate that IL-21 is involved in modulating EAE progression and this pathway be mediated by upregulation of IL-10.

The contents of all references, pending patent applications and published patents, cited throughout this application are hereby expressly incorporated by reference.

### Equivalents

Those skilled in the art will recognize, or be able to ascertain using no more than routine experimentation, many equivalents of the specific embodiments of the invention described herein. Such equivalents are intended to be encompassed by the following claims.

What is claimed is:

1. A method of ameliorating a symptom of multiple sclerosis in a subject, the method comprising:
administering to the subject an agonist of an interleukin-21 (IL-21)/IL-21 receptor (IL-21 R), in an amount sufficient to ameliorate a symptom of multiple sclerosis, wherein said agonist is selected from the group consisting of an IL-21 polypeptide, an agonistic anti-IL21R antibody and an antigen-binding fragment of an agonistic anti-IL21R antibody.

2. The method of claim 1, wherein the agonist is an IL-21 polypeptide that comprises a sequence at least 90% identical to the amino acid sequence of SEQ ID NO:2 and is capable of binding to an IL-21R.

3. The method of claim 1, wherein the agonist is an IL-21 polypeptide that comprises a sequence at least 95% identical to the amino acid sequence of SEQ ID NO:2 and is capable of binding to an IL-21R.

4. The method of claim 1, wherein the agonist is an IL-21 polypeptide that comprises the amino acid sequence of SEQ ID NO:2.

5. The method of claim 1, wherein the agonist is an agonistic anti-IL21R antibody or an antigen-binding fragment thereof.

6. The method of claim 5, wherein the agonistic anti-IL21R antibody is a human antibody.

7. The method of claim 1, further comprising administering to the subject at least one anti-inflammatory agent.

8. The method of claim 7, wherein the anti-inflammatory agent is selected from the group consisting of IFN β-1α, IFNβ-1β, TNF antagonists, IL-12 antagonists, IL-23 antagonists, methotrexate, leflunomide, sirolimus (rapamycin), and CCI-779.

9. The method of claim 1, wherein the subject is a mammal.

10. The method of claim 1, wherein the IL-21/IL-21R agonist is administered in the form of a single dose.

11. The method of claim 1, wherein the IL-21/IL-21R agonist is administered as a series of doses separated by intervals of days, weeks or months.

12. The method of claim 1, wherein the IL-21/IL-21R agonist is administered by injection.

13. The method of claim 12, wherein the IL-21/IL-21R agonist is injected into the central nervous system.

14. The method of claim 12, wherein the IL-21/IL-21R agonist is injected intrathetically or intravenously.

15. The method of claim 12, wherein the IL-21/IL-21R agonist is injected into the lumbar cerebrospinal fluid.

16. The method of 1, further comprising evaluating a subject for risk of multiple sclerosis by evaluating an IL-10 parameter of the subject.

17. The method of 1, further comprising, prior to the administering, evaluating an IL-10 parameter of the subject.

18. The method of 17, further comprising, after to the administering, evaluating an IL-10 parameter of the subject, wherein an increase in the IL-10 parameter indicates a therapeutic effect.

19. The method of 1, further comprising, after to the administering, evaluating an IL-10 parameter of the subject.

20. A pharmaceutical composition comprising an IL-21/IL-21R agonist and an anti-inflammatory agent, wherein said IL-21/IL-21R agonist is selected from the group consisting of an IL-21 polypeptide, an agonistic anti-IL21R antibody and an antigen-binding fragment of an agonistic anti-IL21R antibody.

21. The pharmaceutical composition of claim 20, wherein the IL-21 polypeptide has a sequence at least 90% identical to the amino acid sequence of SEQ ID NO:2 and is capable of binding to an IL-21R.

22. The pharmaceutical composition of claim 20, wherein the IL-21 polypeptide has a sequence at least 95% identical to the amino acid sequence of SEQ ID NO:2 and is capable of binding to an IL-21R.

23. The pharmaceutical composition of claim 20, wherein said IL-21/IL-21R agonist comprises the amino acid sequence of SEQ ID NO:2.

24. The pharmaceutical composition of claim 20, wherein the anti-inflammatory agent is selected from the group consisting of IFN β-1α, IFNβ-1β, TNF antagonists, IL-12 antagonists, IL-23 antagonists, methotrexate, leflunomide, sirolimus (rapamycin), and CCI-779.

25. The pharmaceutical composition of claim 20, wherein the agonist is an agonistic anti-IL21R antibody or an antigen-binding fragment thereof.

26. The pharmaceutical composition of claim 25, wherein the agonistic anti-IL21R antibody is a human antibody.

27. A pharmaceutical composition comprising an IL-21/IL-21R agonist and a protein that simulates myelin basic protein, wherein said IL-21/IL-21R agonist is selected from the group consisting of an IL-21 polypeptide, an agonistic anti-IL21R antibody and an antigen-binding fragment of an agonistic anti-IL21 R antibody.

28. The pharmaceutical composition of claim 27 wherein the IL-21/IL-21R agonist is a protein that comprises an IL-21 polypeptide, and the protein that simulates myelin basic protein comprises glatiramer acetate.

29. A method of ameliorating multiple sclerosis in a mammalian subject, the method comprising:
administering to the subject an interleukin-21 (IL-21) polypeptide in an amount sufficient to ameliorate multiple sclerosis, or at least one symptom of multiple sclerosis in the subject.

30. The method of claim 29 wherein the subject is human, and the IL-21 polypeptide is a human IL-21 polypeptide.

31. The method of claim 30 wherein the IL-21 polypeptide comprises SEQ ID NO:2.

32. The method of claim 30 wherein the IL-21 polypeptide is recombinantly produced.

33. The method of claim 30 wherein the IL-21 polypeptide is recombinantly produced in a bacterial cell.

34. A method of modulating an IL-10 deficiency, or a disorder associated with an IL-10 deficiency in a mammalian subject, the method comprising:
administering to the subject an interleukin-21 (IL-21) polypeptide in an amount sufficient to increase IL-10 expression or activity in the subject.

35. A method of treating or preventing an immunological disorder in a mammalian subject, the method comprising:
evaluating an IL-10 parameter in a mammalian subject; and
administering, to the subject, an interleukin-21 (IL-21) polypeptide in an amount that is dependent on results of the evaluated IL-10 parameter.

36. The method of claim 35 wherein the IL-10 parameter comprises quantitative information about levels of IL-10 protein or IL-10 mRNA.

37. The method of claim 35 wherein the IL-10 parameter comprises quantitative information about levels of IL-10 protein activity.

38. The method of claim 35 wherein the immunological disorder is a neurological disorder.

39. The method of claim 38 wherein the subject is human and the immunological disorder is multiple sclerosis.

40. The method of claim 38 wherein the immunological disorder causes damage or alteration to myelin sheaths.

41. A method of evaluating treatment of multiple sclerosis in a mammalian subject, the method comprising:
administering, to the subject, an agonist of an interleukin-21 (IL-21)/IL-21 receptor (IL-21R); and
evaluating an IL-10 parameter in the subject.

42. The method of claim 41 further comprising administering to the subject a second dose of the agonist, wherein the second dose is administered as a function of the evaluated IL-10 parameter.

43. The method of claim 41 wherein the agonist is selected from the group consisting of an IL-21 polypeptide, an agonistic anti-IL21R antibody and an antigen-binding fragment of an agonistic anti-IL21R antibody.

44. The method of claim 43 wherein the agonist is an IL-21 polypeptide.

45. The method of claim 44 wherein the subject is human, and the IL-21 polypeptide is a human IL-21 polypeptide.

46. The method of claim 44 wherein the IL-21 polypeptide comprises SEQ ID NO:2.

47. An article of manufacture comprising
(i) a container with one or more unit doses of a pharmaceutical composition comprising an IL-21 polypeptide; and
(ii) instructions for administering the unit doses to a subject that has, or is suspected of having, multiple sclerosis.

48. The article of 47 wherein the instructions are provided on a label.

49. The article of 48 wherein the label is affixed to an external surface of the container.


SEQUENCE LISTING (13 sequences)

SEQ ID NO: 1 - DNA, 617 bp, Homo sapiens
gctgaagtgaaaacgagaccaaggtctagctctactgttggtacttatgagatccagtcctggcaacatggagaggattgtcatctgtctgatggtcatcttcttggggacactggtccacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattcctgaggatctaacttgcagttggacactatgttacatactctaatatagtagtgaaagtcatttctttgtattccaagtggaggag

SEQ ID NO: 2 - protein, 131 aa, Homo sapiens
QDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 3 - DNA, 3072 bp, Mus musculus
gagaaccagaccaaggccctgtcatcagctcctggagactcagttctggtggcatggagaggacccttgtctgtctggtagtcatcttcttggggacagtggcccataaatcaagcccccaagggccagatcgcctcctgattagacttcgtcaccttattgacattgttgaacagctgaaaatctatgaaaatgacttggatcctgaacttctatcagctccacaagatgtaaaggggcactgtgagcatgcagcttttgcctgttttcagaaggccaaactcaagccatcaaaccctggaaacaataagacattcatcattgacctcgtggcccagctcaggaggaggctgcctgccaggaggggaggaaagaaacagaagcacatagctaaatgcccttcctgtgattcgtatgagaaaaggacacccaaagaattcctagaaagactaaaatggctccttcaaaagatgattcatcagcatctctcctagaacacataggacccgaagattcctgaggatccgagaagattcccgaggactgaggagacgccggacactatagacgctcacgaatgcaggagtacatcttgcctcttgggattgcaagtggagaagtacgatacgttatgataagaacaactcagaaaagctataggttaagatcctttcgcccattaactaagcagacattgtggttccctgcacagactccatgctgtcaacatggaaaatctcaactcaacaagagcccagcttcccgtgtcagggatttctggtgcttctcaagctgtggcttcatcttattgcccaactgtgacattctttgattggaaggggaaaactaaagcttttagcaaaaatacagctagggaatttgtcgatctgcgagagtaagacctcttatgatcctaacggaatgatgtaagctggaaataataagcataagatgaaattgaaaattgaagtctttattctttaagaaaaactttgtacttgaaagcatgtctgaagagtttactcattaccacaaacatctagcatattgataactaacatctttatactctacaagagaggctttccagataggtacagtttttcttctctattaggtctatcaaaatttaacctattatgagggtcacccctggctttcactgtttttctaaagaggcaagggtgtagtaagaagcaggcttaagttgccttcctcccaatgtcaagttcctttataagctaatagtttaatcttgtgaagatggcaatgaaagcctgtggaagtgcaaacctcactatcttctggagccaagtagaattttcaagtttgtagctctcacctcaagtggttatgggtgtcctgtgatgaatctgctagctccagcctcagtctcctctcccacatcctttcctttctttcctctttgaaacttctaagaaaaagcaatccaaacaagttcagcacttaagacacattgcatgcacacttttgataagttaaatccaaccatctatttaaaatcaaaatcaggagatgagccaagagaccagaggttctgttccagttttaaacagacttttactgaacatcccaatcttttaaccacagaggctaaattgagcaaatagttttgccatttgatataatttccaacagtatgtttcaatgtcaagttaaaaagtctacaaagctattttccctggagtggtatcatcgctttgagaatttcttatggttaaaatggatctgagatccaagcatggcctgggggatggttttgatctaaggaaaaaggtgtctgtacctcacagtgcctttaaaacaagcagagatcccgtgtaccgccctaagatagcacagactagtgttaactgattcccagaaaagtgtcacaatcagaaccaacgcattctcttaaactttaaaaatatgtattgcaaagaacttgtgtaactgtaaatgtgtgactgttgatgacattatacacacatagcccacgtaagtgtccaatggtgctagcattggttgctgagtttgctgctcgaaagctgaagcagagatgcagtccttcacaaagcaatgatggacagagaggggagtctccatgttttattcttttgttgtttctggctgtgtaactgttgacttcttgacattgtgatttttatatttaagacaatgtatttattttggtgtgtttattgttctagccttttaaatcactgacaatttctaatcaagaagtacaaataattcaatgcagcacaggctaagagcttgtatcgtttggaaaagccagtgaaggcttctccactagccatgggaaagctacgctttagagtaaactagacaaaattgcacagcagtcttgaacctctctgtgctcaagactcagccagtcctttgacattattgttcactgtgggtgggaacacattggacctgacacactgttgtgtgtccatgaaggttgccactggtgtaagctttttttggttttcattctcttatctgtagaacaagaatgtggggctttcctaagtctattctgtattttattctgaacttcgtatgtctgagttttaatgttttgagtactcttacaggaacacctgaccacacttttgagttaaattttatcccaagtgtgatatttagttgttcaaaaagggaagggatatacatacatacatacatacatacatacatatatatatatatatatacatatatatatatatatatatgtatatatatatatatatagagagagagagagagagagagagagaaagagagagaggttgttgtaggtcataggagttcagaggaaatcagttatggccgttaatactgtagctgaaagtgttttctttgtgaataaattcatagcattattgatctatgttattgctctgttttatttacagtcacacctgagaatttagttttaatatgaatgatgtactttataacttaatgattatttattatgtatttggttttgaatgtttgtgttcatggcttcttatttaagacctgatcatattaaatgctacccagtccgga

SEQ ID NO: 4 - protein, 122 aa, Mus musculus
PDRLLIRLRHLIDIVEQLKIYENDLDPELLSAPQDVKGHCEHAAFACFQKAKLKPSNPGNNKTFIIDLVAQLRRRLPARRGGKKQKHIAKCPSCDSYEKRTPKEFLERLKWLLQKMIHQHLS

SEQ ID NO: 5 - DNA, 2665 bp, Homo sapiens
gtcgactggaggcccagctgcccgtcatcagagtgacaggtcttatgacagcctgattggtgactcgggctgggtgtggattctcaccccaggcctctgcctgctttctcagaccctcatctgtcacccccacgctgaacccagctgccacccccagaagcccatcagactgcccccagcacacggaatggatttctgagaaagaagccgaaacagaaggcccgtgggagtcagcatgccgcgtggctgggccgcccccttgctcctgctgctgctccagggaggctggggctgccccgacctcgtctgctacaccgattacctccagacggtcatctgcatcctggaaatgtggaacctccaccccagcacgctcacccttacctggcaagaccagtatgaagagctgaaggacgaggccacctcctgcagcctccacaggtcggcccacaatgccacgcatgccacctacacctgccacatggatgtattccacttcatggccgacgacattttcagtgtcaacatcacagaccagtctggcaactactcccaggagtgtggcagctttctcctggctgagagcatcaagccggctccccctttcaacgtgactgtgaccttctcaggacagtataatatctcctggcgctcagattacgaagaccctgccttctacatgctgaagggcaagcttcagtatgagctgcagtacaggaaccggggagacccctgggctgtgagtccgaggagaaagctgatctcagtggactcaagaagtgtctccctcctccccctggagttccgcaaagactcgagctatgagctgcaggtgcgggcagggcccatgcctggctcctcctaccaggggacctggagtgaatggagtgacccggtcatctttcagacccagtcagaggagttaaaggaaggctggaaccctcacctgctgcttctcctcctgcttgtcatagtcttcattcctgccttctggagcctgaagacccatccattgtggaggctatggaagaagatatgggccgtccccagccctgagcggttcttcatgcccctgtacaagggctgcagcggagacttcaagaaatgggtgggtgcacccttcactggctccagcctggagctgggaccctggagcccagaggtgccctccaccctggaggtgtacagctgccacccaccacggagcccggccaagaggctgcagctcacggagctacaagaaccagcagagctggtggagtctgacggtgtgcccaagcccagcttctggccgacagcccagaactcggggggctcagcttacagtgaggagagggatcggccatacggcctggtgtccattgacacagtgactgtgctagatgcagaggggccatgcacctggccctgcagctgtgaggatgacggctacccagccctggacctggatgctggcctggagcccagcccaggcctagaggacccactcttggatgcagggaccacagtcctgtcctgtggctgtgtctcagctggcagccctgggctaggagggcccctgggaagcctcctggacagactaaagccaccccttgcagatggggaggactgggctgggggactgccctggggtggccggtcacctggaggggtctcagagagtgaggcgggctcacccctggccggcctggatatggacacgtttgacagtggctttgtgggctctgactgcagcagccctgtggagtgtgacttcaccagccccggggacgaaggacccccccggagctacctccgccagtgggtggtcattcctccgccactttcgagccctggaccccaggccagctaatgaggctgactggatgtccagagctggccaggccactgggccctgagccagagacaaggtcacctgggctgtgatgtgaagacacctgcagcctttggtctcctggatgggcctttgagcctgatgtttacagtgtctgtgtgtgtgtgtgcatatgtgtgtgtgtgcatatgcatgtgtgtgtgtgtgtgtgtcttaggtgcgcagtggcatgtccacgtgtgtgtgtgattgcacgtgcctgtgggcctgggataatgcccatggtactccatgcattcacctgccctgtgcatgtctggactcacggagctcacccatgtgcacaagtgtgcacagtaaacgtgtttgtggtcaacagatgacaacagccgtcctccctcctagggtcttgtgttgcaagttggtccacagcatctccggggctttgtgggatcagggcattgcctgtgactgaggcggagcccagccctccagcgtctgcctccaggagctgcaagaagtccatattgttccttatcacctgccaacaggaagcgaaaggggatggagtgagcccatggtgacctcgggaatggcaattttttgggcggcccctggacgaaggtctgaatcccgactctgataccttctggctgtgctacctgagccaagtcgcctcccctctctgggctagagtttccttatccagacagtggggaaggcatgacacacctgggggaaattggcgatgtcacccgtgtacggtacgcagcccagagcagaccctcaataaacgtcagcttccttcaaaaaaaaaaaaaaaaatctaga

SEQ ID NO: 6 - protein, 538 aa, Homo sapiens
MPRGWAAPLLLLLLQGGWGCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 7 - DNA, 2628 bp, Mus musculus
gtcgacgcggcggtaccagctgtctgcccacttctcctgtggtgtgcctcacggtcacttgcttgtctgaccgcaagtctgcccatccctggggcagccaactggcctcagcccgtgccccaggcgtgccctgtctctgtctggctgccccagccctactgtcttcctctgtgtaggctctgcccagatgcccggctggtcctcagcctcaggactatctcagcagtgactcccctgattctggacttgcacctgactgaactcctgcccacctcaaaccttcacctcccaccaccaccactccgagtcccgctgtgactcccacgcccaggagaccacccaagtgccccagcctaaagaatggctttctgagaaagaccctgaaggagtaggtctgggacacagcatgccccggggcccactggctgccttactcctgctgattctccatggagcttggagctgcctggacctcacttgctacactgactacctctggaccatcacctgtgtcctggagacacggagccccaaccccagcatactcagtctcacctggcaagatgaatatgaggaacttcaggaccaagagaccttctgcagcctacacaggtctggccacaacaccacacatatatggtacacgtgccatatgcgcttgtctcaattcctgtccgatgaagttttcattgtcaatgtgacggaccagtctggcaacaactcccaagagtgtggcagctttgtcctggctgagagcatcaaaccagctccccccttgaacgtgactgtggccttctcaggacgctatgatatctcctgggactcagcttatgacgaaccctccaactacgtgctgaggggcaagctacaatatgagctgcagtatcggaacctcagagacccctatgctgtgaggccggtgaccaagctgatctcagtggactcaagaaacgtctctcttctccctgaagagttccacaaagattctagctaccagctgcaggtgcgggcagcgcctcagccaggcacttcattcagggggacctggagtgagtggagtgaccccgtcatctttcagacccaggctggggagcccgaggcaggctgggaccctcacatgctgctgctcctggctgtcttgatcattgtcctggttttcatgggtctgaagatccacctgccttggaggctatggaaaaagatatgggcaccagtgcccacccctgagagtttcttccagcccctgtacagggagcacagcgggaacttcaagaaatgggttaatacccctttcacggcctccagcatagagttggtgccacagagttccacaacaacatcagccttacatctgtcattgtatccagccaaggagaagaagttcccggggctgccgggtctggaagagcaactggagtgtgatggaatgtctgagcctggtcactggtgcataatccccttggcagctggccaagcggtctcagcctacagtgaggagagagaccggccatatggtctggtgtccattgacacagtgactgtgggagatgcagagggcctgtgtgtctggccctgtagctgtgaggatgatggctatccagccatgaacctggatgctggccgagagtctggccctaattcagaggatctgctcttggtcacagaccctgcttttctgtcttgcggctgtgtctcaggtagtggtctcaggcttggaggctccccaggcagcctactggacaggttgaggctgtcatttgcaaaggaaggggactggacagcagacccaacctggagaactgggtccccaggagggggctctgagagtgaagcaggttccccccctggtctggacatggacacatttgacagtggctttgcaggttcagactgtggcagccccgtggagactgatgaaggaccccctcgaagctatctccgccagtgggtggtcaggacccctccacctgtggacagtggagcccagagcagctagcatataataaccagctatagtgagaagaggcctctgagcctggcatttacagtgtgaacatgtaggggtgtgtgtgtgtgtgtgtgtgtgtgtgtgtgtgtgtgtgtgtgtgtgtgtgtgtgtgtgtcttgggttgtgtgttagcacatccatgttgggatttggtctgttgctatgtattgtaatgctaaattctctacccaaagttctaggcctacgagtgaattctcatgtttacaaacttgctgtgtaaaccttgttccttaatttaataccattggttaaataaaattggctgcaaccaattactggagggattagaggtagggggcttttgagttacctgtttggagatggagaaggagagaggagagaccaagaggagaaggaggaaggagaggagaggagaggagaggagaggagaggagaggagaggagaggagaggagaggagaggctgccgtgaggggagagggaccatgagcctgtggccaggagaaacagcaagtatctggggtacactggtgaggaggtggccaggccagcagttagaagagtagattaggggtgacctccagtatttgtcaaagccaattaaaataacaaaaaaaaaaaaaaagcggccgctctaga

SEQ ID NO: 8 - protein, 529 aa, Mus musculus
MPRGPVAALLLLILHGAWSCLDLTCYTDYLWTITCVLETRSPNPSILSLTWQDEYEELQDQETFCSLHRSGHNTTHIWYTCHMRLSQFLSDEVFIVNVTDQSGNNSQECGSFVLAESIKPAPPLNVTVAFSGRYDISWDSAYDEPSNYVLRGKLQYELQYRNLRDPYAVRPVTKLISVDSRNVSLLPEEFHKDSSYQLQVRAAPQPGTSFRGTWSEWSDPVIFQTQAGEPEAGWDPHMLLLLAVLIIVLVFMGLKIHLPWRLWKKIWAPVPTPESFFQPLYREHSGNFKKWVNTPFTASSIELVPQSSTTTSALHLSLYPAKEKKFPGLPGLEEQLECDGMSEPGHWCIIPLAAGQAVSAYSEERDRPYGLVSIDTVTVGDAEGLCVWPCSCEDDGYPAMNLDAGRESGPNSEDLLLVTDPAFLSCGCVSGSGLRLGGSPGSLLDRLRLSFAKEGDWTADPTWRTGSPGGGSESEAGSPPGLDMDTFDSGFAGSDCGSPVETDEGPPRSYLRQWVVRTPPPVDSGAQSS

SEQ ID NO: 9 - protein, 16 aa, Homo sapiens
MPLLLLLLLLPSPLHP

SEQ ID NO: 10 - protein, 162 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 11 - protein, 5 aa, Artificial
  Synthetic linker
SGGGG

SEQ ID NO: 12 - protein, 122 aa, Bos taurus
QDRLFIRLRQLIDIVDQLKNYVNDLDPEFLPAPEDVKRHCERSAFSCFQKVQLKSANNGDNEKIINILTKQLKRKLPATNTGRRQKHEVTCPSCDSYEKKPPKEYLERLKSLIQKMIHQHLS

SEQ ID NO: 13 - protein, 146 aa, Mus musculus [sequence omitted]